(19) United States
(12) Patent Application Publication
(10) Pub. No.: US 2017/0313746 A1
(43) Pub. Date: Nov. 2, 2017

(54) Peptides Useful For Treating Cancer

(71) Applicant: Hilmar M. Warenius, Heswall, Wirral (GB)

(72) Inventor: Hilmar M. Warenius, Heswall, Wirral (GB)

(21) Appl. No.: 15/501,748

(22) PCT Filed: Aug. 5, 2015
(86) PCT No.: PCT/EP2015/068056

§ 371 Date: Jun. 29, 2017

(30) Foreign Application Priority Data
Aug. 6, 2014 (GB) 1413942.2

(51) Int. Cl. C07K 7/64; A61K 31/7004; A61K 38/12; A61K 38/00

(52) U.S. Cl. CPC C07K 7/64; A61K 38/12; A61K 31/7004; A61K 38/00

(57) ABSTRACT

Provided is a class of peptides which are useful for modulating the activity of poly (ADP-ribose) polymerase (PARP) and in particular for the treatment of cancer. The peptides include an active group and a cassette for delivering the active group to a cell. Also provided are peptides having an anionic group which is believed to act as a competitive inhibitor of proteases which cleave PARP.

This application claims priority from GB patent application no. 1413942.2, filed on 6 Aug. 2014. The contents of the priority document are incorporated herein by reference in their entirety.

### FIELD OF THE INVENTION

The present invention relates to peptides and peptide mimetics useful for the treatment of cancer, and in particular to peptides and mimetic compounds which selectively cause cancer cell necrosis accompanied by ATP depletion.

### BACKGROUND OF THE INVENTION

The main thrust in anticancer drug development at the present time derives from the explosion in knowledge of cell surface receptors and positive and negative signal transduction factors, recently further fuelled by genomic studies of several common human cancers [Pleasance et al. Nature (2009) 463: 191-196; Sjöblom et al. Science (2006) 314:268-274; Greenman et al. Nature (2007) 446:153-158; Jones et al. Science (2008) 321:1801-1806; Gerlinger et al. (2012) 366:883-892]. These studies have revealed a multitude of genetic mutations, hundreds of which are believed to be driver mutations involving critical proteins on signal transduction pathways that contribute to the evolution of autonomous cancer cell proliferation.

A multiplicity of potential drug targets are being revealed by this approach, with an even greater number of potential therapeutic agents, as several different drugs may show activity against any one target.

The present anticancer therapeutic paradigm envisages progress towards tailored drug treatment for individually selected cancers on the basis of their genomic mutation patterns. The resulting therapeutics are being rapidly introduced into the clinic. These new drugs, however, have generally poor single agent efficacy, with very few complete tumour responses, and median response durations of less than a year in the majority of cases.

There is thus a need for more global anticancer therapeutic agents.

In contrast to the multiplicity and heterogeneity of mutation-derived signal transduction targets, certain generalised abnormalities, such as aerobic glycolysis and aneuploidy, have been observed in cancer cells for many years. These changes remain potential global “Achilles heels” for therapeutic exploitation.

Aerobic glycolysis was first described by Otto Warburg [Warburg et al. J Gen Physiol (1927) 8:519-530] as a generalised difference between cancer cells and normal cells. He identified increased uptake of glucose and production of lactate, characteristic of aerobic glycolysis in cancer cells even in the presence of adequate oxygen. This finding, which suggests abnormal carbohydrate metabolism in cancer cells as compared to normal, could provide a global anticancer target and continues to be actively researched [Reviewed by Dang et al. J Mol Med (2011) 89:205-212].

Two key molecular sites in which carbohydrate metabolism in cancer cells can be therapeutically targeted are the enzymes hexokinase 2 and lactate dehydrogenase.

Hexokinase 2 phosphorylates glucose following its uptake through the cell membrane, thus trapping the glucose intracellularly for glycolysis. The importance of hexokinase 2 (HK2) as a potentially selective systemic cancer target has recently been highlighted by Hk2 deletion experiments in mice [Ros and Schulze Cancer Discov; (2013) 3:1105-1107]. Hexokinase 2 inhibition as an anticancer treatment has been attempted in vivo in mouse xenograft models [Xu et al. Cancer Res; (2005) 65:613-621]. Although a weak tumour inhibitor on its own, 2-deoxyglucose has been shown to be effective when used in combination with metformin against a broad spectrum of preclinical cancer models [Cheong et al. Mol Cancer Ther (2011) 10:2350-2362]. A further cancer therapeutic inhibitor of hexokinase 2 is 3-bromopyruvate [Ko et al. Cancer Lett (2001) 173:83-91] but this has problems of normal tissue toxicity.

Lactate dehydrogenase A (LDHA) has been known to be elevated in tumours for many years and has been identified as a direct target of the c-Myc oncogenic transcription factor [Le et al. PNAS (2010) 107:2037-2042]. Medicinal chemistry programmes to design inhibitors of LDHA as anticancer therapeutics are presently underway [Granchi et al. J. Med Chem (2011) 54:1599-1612].

In addition to disordered glycolysis, energy levels in cancer cells are also influenced by the activity of poly-ADP-ribose polymerase.

Poly (ADP-ribose) polymerase-1 [PARP-1] is the principal member of a family of enzymes possessing poly (ADP-ribosylation) catalytic activity (Munoz-Gamez et al., Biochem J (2005); 386: 119-125). It consists of three conserved major domains: an NH₂-terminal DNA-damage sensing and binding domain containing three zinc fingers, an automodification domain, and a C-terminal catalytic domain (Javle and Curtin, Brit J Cancer (2011): 105: 114-122).

PARP-1 is a chromatin-associated, conserved, nuclear protein (Cherney et al.; Proc. Natl Acad. Sci. USA. 1987; 84:8370-8374) that has the capacity to bind rapidly and directly to both single- and double-strand DNA breaks. Both types of DNA breakage activate the catalytic capacity of the enzyme, which in turn modulates the activity of a wide range of nuclear proteins by covalent attachment of branching chains of ADP-ribose moieties (Munoz-Gamez et al., Biochem J (2005); 386: 119-125). A principal function of the poly ADP-ribose chains is to alert repair enzymes to sites of DNA damage.

When PARP-1 is activated by DNA breaks, it cleaves NAD⁺ (nicotinamide adenine dinucleotide) to generate nicotinamide and the ADP-ribose which foul's the chains that attach to DNA adjacent to strand breaks (Javle and Curtin, Brit J Cancer (2011) 105:114-122). The cleavage of NAD⁺ by PARP to form ADP-ribose chains on DNA results in less NAD⁺ being available to generate ATP, which is an essential energy source for the cell. Thus, PARP activity can lead to a drop in cellular ATP levels.

Apoptosis is active “cell suicide” which is an energy-dependent process. Depletion of ATP as a result of PARP activity can deprive the cell of the requisite energy to carry out apoptosis. An important component of a successful apoptotic process is thus cleavage of PARP to prevent ATP depletion. Cleavage inactivates poly-(ADP-ribosylation) and is carried out by several caspases, especially caspase-3 (Herceg and Wang, Mol Cell Biol (1999); 19:5124-5133). Caspase-3 cleaves the 113-kDa PARP protein at the DEVD site [Gly-Asp-Glu-Val-Asp₂₁₄-Gly₂₁₅ (SEQ ID NO: 1)] between Asp 214 and Gly 215 amino acids to yield two fragments, an 89- and a 24-kDa polypeptide.

The cleavage fragments from PARP appear to contribute to the suppression of PARP activity, because p89 and p24 inhibit homo-association and DNA binding of intact PARP respectively (Graziani and Szabo 2005, Pharmacol Res. (2005); 52:109-118).

Whereas high levels of ATP enable cells to undergo apoptosis, low levels of ATP shift cells away from apoptosis towards necrosis (Eguchi Y, Shimizu S, Tsujimoto Y, Cancer Res (1997); 57:1835-1840). PARP has been shown to be a mediator of necrotic death by ATP depletion in mouse fibroblasts. Fibroblasts from PARP-deficient mice (PARP−/−) are protected from ATP depletion and necrotic death (Ha and Snyder 1999, Proc Natl Acad Sci (1999): 96:13978-13982).

In summary, PARP is a 113-kDa protein which flags DNA breaks with poly ADP-ribose chains for recognition by repair enzymes. The poly ADP-ribose is formed by breakdown of NAD which can lead to depletion of the ATP necessary for apoptosis and potentially result in cell death by necrosis.

Aneuploidy is another global change which is characteristic of cancer cells and absent in normal cells [Duesberg and Rasnik. Cell Motility and the Cytoskeleton (2000) 47:81-107]. Aneuploidy is strictly defined as an aberrant chromosome number that deviates from a multiple of the haploid number of chromosomes found in normal cells [Holland and Cleveland EMBO reports (2012) 13: 501-514].

A considerable body of work has been directed towards the question of whether aneuploidy is an intrinsic component of the cause of malignant transformation of normal cells, or the result of the genetic instability which frequently accompanies this malignant change [Li PNAS (2000) 97:3236-3241; Knaus and Klein J Biosci (2012) 37:211-220]. A key point is, however, that aneuploidy is a manifestation of the marked DNA damage that is found in cancer cells, as a parallel consequence either of abnormal mitosis preceding aneuploidy [Ganem and Pellman J Cell Biol (2012) 199: 871-881] or of segregative errors of aneuploid chromosomes [Jenssen et al. Science 92011) 333:1895-1898].

A clear difference between cancer cells and normal cells is that cancer cells with severely damaged genomes have a much greater requirement for DNA repair than do normal cells. A major component of DNA repair processes is the “flagging” of DNA damage by poly (ADP-ribose) polymerase-1 [PARP-1].

It is thus unsurprising that increased PARP activity, as measured by mRNA expression, has been observed in a wide range of different human cancers as compared to the normal tissues from which they have arisen [Ossovskaya et al. Genes and Cancer (2010) 1:812-821].

Cancer cells, therefore, operate at an energy deficit as compared to normal cells, as a result of disordered carbohydrate metabolism and the high energy needs required for repeated cell doublings and the repair of their massive DNA damage. In addition, the energy needed to accomplish each repeated cancer cell division would be expected to place a further burden on this energy deficit.

There is an, as yet unfulfilled, role for anticancer therapeutics capable of exploiting the above global energy-deficit target present in cancer cells but not in normal cells.

Increased PARP activity has been shown to lead to cellular necrosis following ascorbate/menadione-induced oxidative stress causing DNA damage in K562 cells [Verrax et al. Int J Cancer (2007) 120:1192-1197] and in CX cells poisoned by cyanide, in which the caspase cascade was inhibited with zVAD-fink [Prabhakaran et al. Toxicology and Applied Pharmacology (2004) 195:194-202]. In these cases, however, in addition to maintaining PARP function, DNA damage or oxidative stress are also needed for cellular necrosis to occur. The caspase inhibitor zVAD-fink alone did not cause necrosis. Similarly other caspase inhibitors such as survivin [Hensley et al. Biol Chem (2013) 394:831-843] and DEVD-CHO [Coelho et al. Brit J Cancer (2000) 83:642-629] do not on their own cause necrosis. Moreover, small molecule antagonists of XIAP caspase inhibitors stimulate caspase activity but induce apoptosis rather than necrosis [Schimmer et al. Cancer Cell 92004) 5:25-35].

Thus PARP agonists, such as caspase inhibitors, despite maintaining active PARP do not on their own appear to induce cellular necrosis. In addition rendering PARP insensitive to caspase cleavage at the DEVD site by a point mutation did not on its own cause necrosis. Necrosis only occurred when TNF-α was added [Herceg and Wang Molec Cell Biol (1999) 219:5124-5133].

In summary, a number of PARP agonists have been described, none of which cause cellular necrosis on their own but which can cause necrosis in combination with other agents. Here, for the first time PARP agonists are described which can cause cancer cell death, by ATP depletion, on their own without the need for a second agent.

Current attempts to exploit PARP function therapeutically have concentrated on the development of PARP inhibitors that would prevent poly(ADP-ribosylation) and thus potentiate the effect of DNA-damaging therapeutic agents, leading to apoptosis rather than necrosis (Munoz-Gamez et al., Biochem J (2005); 386:119-125; Plummer, Curr. Opin. Pharmacol. (2005); 6:364-368; Graziani and Szabo, Pharmacol Res. (2005); 52:109-118).

One of the first commercial PARP inhibitors was Olaparib (AZD 2281) (4-[3-(4-cyclopropanecarbonylpiperazine-1-carbonyl)-4-fluorobenzyl]-2H-phthalazin-1-one). Menear et al., Journal of Medicinal Chemistry (2008); 51:6581-91). Olaparib has been studied preclinically and clinically as a potential enhancer of the DNA damaging drug Temozolomide (Khan et al., British Journal of Cancer (2011); 104:750-755).

The inclusion of SEQ ID NO: 2 (PRGPRP) within small peptides has been shown to be selectively cancerocidal towards a wide range of human in-vitro cancer cell lines but not normal diploid human keratinocytes, fibroblasts or immortalised MRC5-hTERT cells (Warenius et al. Molecular Cancer (2011); 10:72-88 and WO/2009/112536).

The ubiquitous, selective anticancer activity of these cyclic peptides is reported to be highly dependent on the arginines within the hexapeptide sequence, because alteration of the amino acid sequence to SEQ ID NO: 3 (Pro-Arg-Arg-Pro-Gly-Pro) removes the cancerocidal capacity, as does substituting either of the arginines for L-NG-monomethyl-arginine or glutamic acid.

Given the multiplicity of peptide sequences in the proteome, it is not unlikely that the sequence PRGPRP (SEQ ID NO: 2), or closely analogous sequences, will randomly occur within the peptide chains of several proteins. For example the D-amino acid sequence PRKPRP (SEQ ID NO: 5) can be found in a Jun binding peptide (JBP) [US2007/0060514 A1] and the hexapeptide PRGPRP (SEQ ID NO: 2) can also be found in the deduced amino-acid sequence of the bbc3 gene [WO00/26228; Reimertz et al. Journal Cell Biology (2003) 162:587-598].

The presence of a peptide sequence within a protein does not, however, mean that it is this sequence in particular, as distinct from other amino-acid sequences within the peptide or protein, that is responsible for the specific functional activity of the whole protein. Functionality of a particular amino acid sequence needs to be proven rather than assumed. In the case of the hexapeptide PRGPRP (SEQ ID NO: 2) in CDK4, which is located on an external loop of the protein, this functionality is selective cancer cell killing by necrosis and this activity is removed by specific alterations in PRGPRP (SEQ. ID NO: 2) such as changing the sequence to PRRPGP (SEQ ID NO: 3) or by N-mono-methylation in the guanidium region of either arginine. There is no specific experimental evidence of functionality, however, for the PRKPRP (SEQ ID NO: 5) region of JBP or the PRGPRP (SEQ ID NO: 2) region of BBC3. Moreover, the whole JPB molecule protects normal neuronal cells against ischaemic necrosis. This is the opposite activity to the CDK4-derived PRGPRP-based cyclic peptide which produces necrosis. In addition, although BBC3 contains a PRGPRP sequence (SEQ ID NO: 2), the whole protein causes apoptosis in normal neurones by interfering with the function of members of the BCL anti-apoptotic protein family. Neither JBP nor BBC3 has been shown to cause selective necrosis of cancer cells as compared to normal, even though they contain a closely homologous or identical sequence to PRGPRP (SEQ ID NO: 2).

Previously described cyclic peptides (WO/2009/112536) were composed of an active PRGPRP site (SEQ ID NO: 2) (“warhead”) and a “backbone” forming a 16-18 amino-acid cyclic peptide of similar dimensions to the externalised loop in CDK4 which contained the PRGPRP amino acid sequence (SEQ ID NO: 2).

The PRGPRP (SEQ ID NO: 1) “warhead” is itself, amphiphilic. If combined in cyclic peptides with non-amphiphilic amino-acid sequences in the “backbone”, the resulting cyclic peptides were inactive [Warenius et al. Molecular Cancer (2011); 10:72-88] viz:

| SEQ ID NO: 6: | |
|---|---|
| Cyc-[AAAGGGPRGPRPGGGAAA] | INACTIVE |
| SEQ ID NO: 7: | |
| Cyc-[GGGGGGPRGPRPGGGGGG] | INACTIVE |
| SEQ ID NO: 8: | |
| Cyc-[GGGGGGPRGPRPGGGGGG] | INACTIVE |
| SEQ ID NO: 9: | |
| Cyc-[AAGPGGPRGPRPGGPGAA] | INACTIVE |

By contrast, the introduction of an amphiphilic, ALKLALKLAL “backbone” (SEQ ID NO: 10), successfully produced active PRGPRP cyclic peptides.

Small differences in the length and composition of amphiphilic “backbones”, however, could make large differences in bio-activity. Thus with regard to killing NCI-H460 human non-small cell lung cancer cells closely similar cyclic peptides demonstrated opposite activities. Viz:

SEQ ID NO: 11:

| | |
|---|---|
| Cyc-[PRGPRPVKLALKLALKLAL] | (“THR52”) INACTIVE |

SEQ ID NO: 12:

| | |
|---|---|
| Cyc-[PRGPRPVKLALKLALKFP] | (“THR5”) ACTIVE |

SEQ ID NO: 13:

| | |
|---|---|
| Cyc-[PRGPRPVALKLALKLAL] | (“THR54”) ACTIVE |

Without being bound by theory, it is likely that the helical structure of the amphiphilic “backbones” constrain the “warhead” in an optimal conformation for bio-activity. In addition, the precise combination of amino-acid sequences in “backbone” and “warhead” can affect the bioactivity of the whole peptide. Thus optimal “backbone”/“warhead” combinations would be anticipated so that the claimed compounds described here would be expected to work most effectively as integral cyclic peptides.

The cyclic peptides THR53, its analogue THR54 (also referred to here as HILR-001), and THR79 (Cyc-[PRGPRPvalklalkalal] (SEQ ID NO: 14) [Warenius et al. Molecular Cancer (2011); 10:72-88 and WO/2009/112536] selectively killed a wide range of human cancer cell lines, but suffered from the problem of low specific activity with IC₅₀s within the 100-200 μM range. Although exhibiting encouraging anticancer therapeutic potential in vitro, these low specific activities precluded testing in vivo against xenografted human cancers, because the systemic doses required would be higher than was tolerable in the mouse.

There is therefore a need for new cyclic peptides which retain the selective cancer cell killing ability of THR53 and THR54 and which have higher specific activity. There is also a need for further active peptide moieties.

US patent application publication no. 2007/0060514 discloses protein kinase inhibitors and more specifically inhibitors of the protein kinase c-Jun amino terminal kinase.

International patent application publication no. 2006/078503 discloses a method for screening for a PARP activator.

International patent application publication no. 2009/112536 discloses a cyclic peptide which comprises a CDK4 peptide region and a cell-penetrating region.

Warenius et al. (Molecular Cancer 2011, 10-72) disclose the selective anticancer activity of a hexapeptide with sequence homology to a non-kinase domain of Cyclin Dependent Kinase 4.

Liu et al. (Neuropathology and Applied Neurobiology (2010), 36, 211-224) state that the c-Jun N-terminal kinase (JNK) inhibitor XG-102 enhances the neuroprotection of hyperbaric oxygen after cerebral ischaemia in adult rats.

Herceg and Wang (Molecular and Cellular Biology, July 1999, pp. 5124-5133) state that the failure of poly(ADP-ribose) polymerase cleavage by caspases leads to induction of necrosis and enhanced apoptosis.

International patent application publication no. 99/18998 discloses a method of packaging a water-insoluble substance, such as, for example, a drug or other therapeutic or diagnostic agent.

### SUMMARY OF INVENTION

Provided herein is a class of anionic/cationic PARP-dependent agents which kill cancer cells by necrosis accompanied by a fall in ATP levels.

In a first aspect, the present invention provides a cyclic compound according to claim 1. Provided is a cyclic compound capable of modulating the activity of poly(ADP-ribose) polymerase 1 (PARP-1), wherein the compound comprises a moiety according to a Formula 1 or salt, derivative, prodrug or mimetic thereof:

[X1-X2-X3-X4-X3-X4-X3-] Formula 1:

wherein X1 is a peptidic moiety capable of inhibiting the cleavage of PARP-1;
wherein X2 may be absent or present; when X2 is present, X2 is selected from Val or Ser;
wherein one of X3 and X4 is selected from Trp-Trp and Ar1-Ar2;
wherein the other of X3 and X4 is selected from Arg-Arg, Gpa-Gpa, Hca-Hca, and Ar3-Ar4; and
wherein
Hca represents the amino acid residue of homocysteic acid;
Gpa represents the amino acid residue of guanidinophenylalanine;
Ar1, Ar2, Ar3 and Ar4 each represent an amino acid residue having an aryl side chain, wherein the aryl side chains are independently selected from an optionally-substituted napthyl group, an optionally substituted 1,2-dihydronapthyl group, and an optionally-substituted 1,2,3,4-tetrahydronapthyl group; and
Aza represents the amino acid residue of azido-homoalanine.

Particularly preferably, X3 is selected from Trp-Trp and Ar1-Ar2 and X4 is selected from Arg-Arg, Gpa-Gpa, and Hca-Hca.

In a second aspect, the present invention provides a compound capable of modulating the activity of poly(ADP-ribose) polymerase 1 according to claim 30. Provided is a compound capable of modulating the activity of poly(ADP-ribose) polymerase 1, which compound comprises a moiety according to Formula 6:

-Pro-X14-X15-Pro-X16-Pro- Formula 6:

wherein X14 and X16 are each independently selected from an amino acid residue bearing a side-chain, a napthyl group bearing a substituent and a propyl group bearing a substituent, wherein each side-chain or substituent comprises an acidic functional group; and wherein X15 is selected from Gly, Ala, MeGly, and (CH₂)₃.

In a third aspect, the present invention provides a pharmaceutical composition comprising a compound in accordance with the first and/or second aspect of the invention.

In a fourth aspect, the present invention provides compounds and compositions in accordance with any of the first to third aspects of the invention which are for use in medicine. The compounds and compositions may be for use in the treatment of cancer.

In a fifth aspect, the present invention provides a method according to claim 51. Provided is a method for treating cancer which method comprises administering to a patient a compound or composition in accordance with any of the first to third aspects of the present invention.

In a sixth aspect, the present invention provides a method according to claim 57. Provided is a method of analysis, which method comprises: contacting cells with a compound of the first or second aspect of the invention; and detecting the compound.

Further areas of applicability of the present invention will become apparent from the detailed description provided hereinafter. The detailed description and specific examples indicate the preferred embodiments of the invention.

### BRIEF DESCRIPTION OF THE DRAWINGS

The present invention will become more fully understood from the detailed description and the accompanying drawings, in which:

FIG. 1 shows the structure of protected guanidinophenylalanine (Gpa) and of homocysteic acid (Hca) for incorporation into peptides by automated peptide synthesis;

FIG. 2 shows the structure of protected azidohomoalanine and 3-amino-3-(-2-naphthyl)-propionic acid, for incorporation into cyclic peptides by automated peptide synthesis;

FIG. 3 shows IC₅₀ plots (% of control v Log [M]) for HILR-001 (SEQ ID NO: 13), HILR-025 (SEQ ID NO: 15) and HILR-030 (SEQ ID NO: 16), demonstrating the increased activity of the HILR-025 sequence (SEQ ID NO: 15) comprising the WWRRWWRRWW amphiphilic cassette (SEQ ID NO: 17) over HILR-001 and the still further increased activity of HILR-030 having a Trp-Trp-Gpa-Gpa-Trp-Trp-Gpa-Gpa-Trp-Trp (SEQ ID NO: 18) cassette over HILR-025 (SEQ ID NO: 15) and also shown is an IC50 plot for HILR-D-08 (SEQ ID NO: 31);

FIG. 4 shows IC₅₀ plots (% of control v Log [M]) for HILR-D-02 (Cyc-[Pro-Glu-Gly-Pro-Glu-Pro-Val-Trp-Trp-Arg-Arg-Trp-Trp-Arg-Arg-Trp-Trp] (SEQ ID NO: 19) and HILR-D-(Cyc-[Pro-Hca-Gly-Pro-Hca-Pro-Val-Trp-Trp-Arg-Arg-Trp-Trp-Arg-Arg-Trp-Trp]) (SEQ ID NO: 20) which demonstrate that anionic groups in the “warhead” are effective;

FIG. 5 is a PARP standard activity curve (a plot of light output v units of purified PARP enzyme);

FIG. 6 shows the effect of Olaparib and 3-aminobenzamide on PARP activity;

FIG. 7 shows the effect of different concentrations of Olaparib on PARP activity over a 96 hour time course;

FIG. 8 shows an IC₅₀ analysis for Olaparib and Paclitaxel;

FIG. 9 shows the effect of HILR-001 in combination with the PARP inhibitor Olaparib on the NC1-NCI-H460 cells over a 96 hour time course. Olaparib partially reverses the HILR-001-induced fall in ATP and consequently reduces the degree of cancer cell necrosis;

FIG. 10 shows the dose response of caspase-3 to Ac-DEVD-CHO;

FIG. 11 shows the effects of Ac-DEVD-CHO and HILR-030 on caspase-3 activity;

FIG. 12 further illustrates the effects of Ac-DEVD-CHO and HILR-030 on caspase-3 activity;

FIG. 13 shows the alignment of the PRGPRP (SEQ ID NO: 2) region of the CDK4 external loop and the DEVD region of PARP and mild but significant killing of NCI-H460 cells by the GDEVDG homologue (HILR-D-01);

FIG. 14 shows peptidomimetic homologues of the cyclic peptides described;

FIG. 15 shows the effects of co-administering 2-deoxyglucose (2-DOG) with cyclic compounds in accordance with the present invention;

FIG. 16 shows morphological changes in NC1 H460 human non-small cell lung cancer cells treated with HILR-025, HILR-D-07, or a DMSO control;

FIG. 17 shows the inhibitory effect of IC₅₀ doses of HILR-025 and HILR-030 on LDH activity at 24 and 96 hours; and

FIG. 18 is a simplified schematic diagram of cellular respiration showing putative sites of action of HILR compounds. Inhibition of LDHA accompanied by an agonistic action on PARP can produce diminished cellular ATP levels. Inhibition of Hexokinase by 6 de-oxy glucose will additionally potentiate the ATP-lowering activity of HILR cyclic peptides.

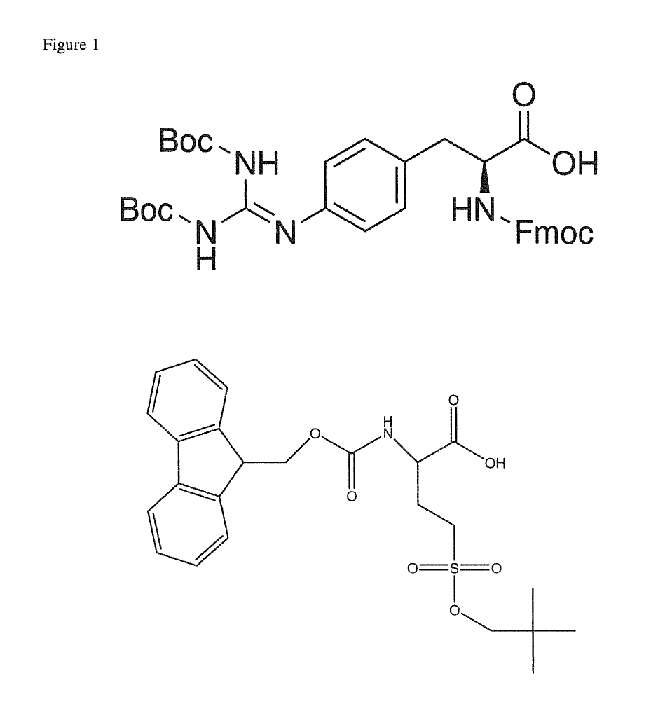

### SEQUENCE LISTING FREE TEXT

SEQ ID NOS: 2, 21, 22, 23, 24, 25, 26, 27, 28, 29, 37, 41 and 42 are cancerocidal groups.

SEQ ID NOS: 3 and 4 are comparative peptides.

SEQ ID NO: 5 is a partial sequence of a Jun binding peptide.

SEQ ID NOS: 6, 7, 8, 9, 11, 12, 13, 14, 15, 16, 19, 20, 30, 31, 32, 33, 34, 35, 36, 39 and 43 to 48 are cyclic peptides.

SEQ ID NOS: 10, 17, 18, 38 and 39 are cassettes.

Some of the appended sequences comprise non-standard unnatural amino acid residues. The unnatural amino acid residues identified in the sequence listing are: guanidinophenylalanine, homocysteic acid, azidohomoalanine, N-methylaspartic acid, the residue of 3-amino-3-(2-napthyl)-propionic acid, and the residue of glutamic acid-gamma-[2-(1-sulfonyl-5-napthyl)-aminoethylamide.

Referring to SEQ ID NO: 21, the free text describing position (2) states “basic residue or an acidic residue selected from homocysteic acid, azidohomoalanine and glutamic acid”. The free text describing position (3) states “selected from Gly, Ala, MeGly, and (CH₂)₃”. The free text describing position (5) states “if residue 2 is acidic, an acidic residue selected from glutamic acid and homocysteic acid. If residue 2 is basic, a basic residue”.

Referring to SEQ ID NO: 24, the free text describing position (2) states “selected from Asp and Glu.” The free text describing position (5) states “selected from Asp, N-alkyl Asp, N-aryl Asp, Glu, N-alkyl Glu, N-Aryl Glu”. The free text describing position (6) states “selected from Gly, N-alkyl Gly, N-aryl Gly”.

Referring to SEQ ID NO: 37, the free text describing position (2) states “any natural or unnatural amino acid bearing an acidic side chain”. The free text describing position (3) states “selected from Gly, Ala, MeGly and (CH₂)₃”. The free text describing position (5) states “any natural or unnatural amino acid bearing an acidic side-chain”.

### DETAILED DESCRIPTION

The present disclosure provides compounds capable of modulating the activity of poly (ADP-ribose) polymerase 1. The compounds may increase the overall poly(ADP-ribose) polymerase 1 activity within a given cell. The compounds may prevent the cleavage of PARP-1 by caspases, and in particular caspase 3. As will be discussed in more detail in the Examples, the compounds provided herein are also believed to inhibit aerobic glycolysis in cancer cells. Cyclic compounds in accordance with the present invention display improved specific activity in comparison to previous cyclic peptides.

The present disclosure provides a cyclic compound capable of modulating the activity of poly(ADP-ribose) polymerase 1 (PARP-1), wherein the compound comprises a moiety according to a Formula 1 or salt, derivative, prodrug or mimetic thereof:

[X1-X2-X3-X4-X3-X4-X3-] Formula 1:
wherein X1 is a peptidic moiety capable of inhibiting the cleavage of PARP-1;
wherein X2 may be absent or present; when X2 is present, X2 is selected from Val or Ser;
wherein one of X3 and X4 is selected from Trp-Trp, and Ar1-Ar2;
wherein the other of X3 and X4 is selected from Arg-Arg, Gpa-Gpa, Hca-Hca, and Ar3-Ar4; and
wherein
   Hca represents the amino acid residue of homocysteic acid;
   Gpa represents the amino acid residue of guanidinophenylalanine;
   Ar1 and Ar2 each represent an amino acid residue having an aryl side chain, wherein the aryl side chains are each independently selected from an optionally-substituted napthyl group, an optionally substituted 1,2-dihydronapthyl group, and an optionally substituted 1,2,3,4-tetrahydronapthyl group; and
   Aza represents the amino acid residue of azido-homoalanine.

Particularly preferably, X3 is selected from Trp-Trp and Ar1-Ar2 and X4 is selected from Arg-Arg-, Gpa-Gpa, Hca-Hca, and Ar3-Ar4.

Throughout the present disclosure, the abbreviation “Hca” refers to the amino acid residue of homocysteic acid. The abbreviation “Gpa” refers to the amino acid residue of guanidinophenylalanine. “Aza” refers to azidohomoalanine. “Nap” represents the amino acid residue of 3-amino-3-(-2-napthyl)-propionic acid. “Eda” represents the following amino acid residue:

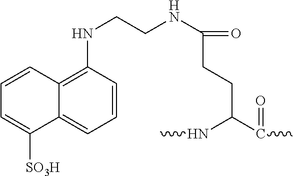

that is, a residue of glutamic acid-gamma-[2-(1-sulfonyl-5-napthyl)-aminoethylamide.

Hca, Gpa, and Aza, along with amino acid residues bearing aryl side chains such as Nap and Eda, are referred to herein as unnatural amino acids. It is preferable to include at least one unnatural amino acid in the compounds of the present disclosure. This is because compounds comprising unnatural amino acids are typically more resistant to degradation by enzymes than compounds consisting of natural amino acids only.

Preferably, the cyclic compound consists of cyclo-[X1-X2-X3-X4-X3-X4-X3] or is a salt, derivative, prodrug or mimetic thereof.

The cyclic compound may comprise a labelling moiety. The labelling moiety may be a fluorescent label.

Labelling moieties allow the detection of the cyclic compound. Examples of labelling moieties include fluorescent labels, radiolabels, mass labels and biotin. Suitable labelling moieties include conventional labels for proteins and peptides. The skilled artisan will be familiar with labels for proteins and peptides.

The labelling moiety may be selected depending on the desired method of detection to be used. For example, if the cyclic compound is to be detected in an ELISA (enzyme-linked immunosorbent assay) then the labelling moiety suitably comprises biotin. In another arrangement, if the cyclic compound is to be detected in a Western blot assay, a gel electrophoresis assay, or the like the labelling moiety is suitably a fluorescent label. Other classes of labels and other assay types are also contemplated herein.

In the arrangements where the cyclic compound comprises Ar1-Ar2 and/or Ar3-Ar4, one or more of the aryl side chains may comprise a substituent, which substituent is a label selected from a fluorescent label, a radiolabel, a mass label, and biotin. Alternatively, one or more of the aryl side chains may comprise a substituent selected such that the aryl side chain functions as a fluorescent label. In this arrangement, the substituent may be a sulfonic acid group. An example of a fluorescent unnatural amino acid comprising an aryl side chain is Eda.

The inclusion of a labelling moiety in the compound may allow the uptake of the compound by a cell to be analysed. The inclusion of labelling moiety may also allow the mechanism of action of the compounds to be elucidated in greater detail. Analysis of cells contacted with labelled compounds may also allow additives, excipients, co-actives, dosages, and dosage forms for inclusion in a formulation comprising the compound to be optimised.

The cyclic compounds disclosed herein comprise an active sequence, often referred to as a “warhead”, and a cassette for delivering the warhead to a cell. X1 represents the active sequence, which is a peptidic moiety capable of inhibiting the cleavage of PARP-1. As used herein, the term peptidic moiety is used to refer to peptide and peptide mimetic moieties. Preferably, X1 is a peptide moiety. It is believed that the active sequences X1 as defined herein either bind to PARP and prevent its cleavage, or competitively inhibit proteases which cleave PARP. PARP is involved in the DNA repair pathway. PARP's mechanism of action consumes NAD leading to ATP depletion. Cancer cells have extensive DNA damage, requiring upregulated PARP activity. Preventing the inactivation of PARP in a cancer cell depletes the cell's ATP, leading to necrosis. Preventing the inactivation of PARP does not deplete a normal cell's ATP, because normal cells have little to no DNA damage. Without being bound by theory, the inventor has discovered that compounds in accordance with the present disclosure therefore selectively cause necrosis in cancer cells by modulating the activity of PARP. It is believed that the compounds may also stress cancer cells by an additional mechanism, further encouraging necrosis. Without wishing to be bound by theory, evidence presented in the Examples suggests that the additional mechanism may relate to the carbohydrate metabolism pathways in cancer cells, specifically the aerobic glycolysis pathway.

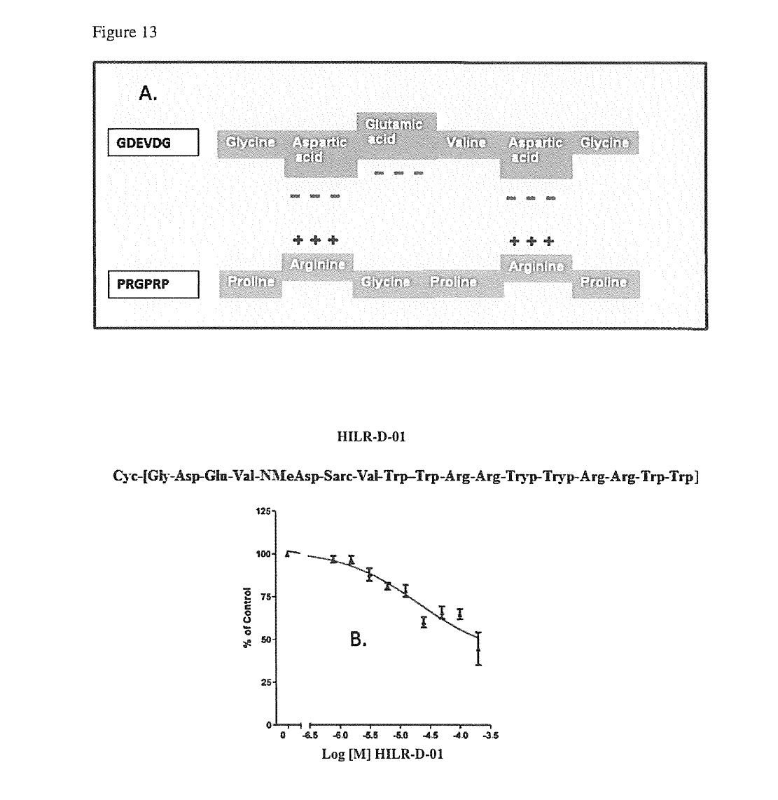

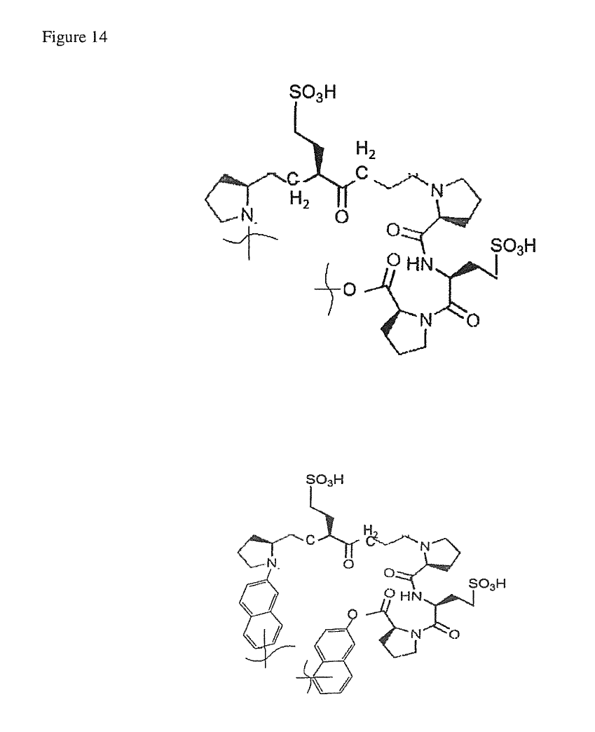

X1 is suitably a moiety which is capable of binding to the DEVD region of PARP. In this arrangement, X1 may be a peptide moiety comprising a total of five or six amino acid residues, preferably 6 amino acid residues. The second and fifth amino acid residues in the sequence may be basic amino acid residues. The basic amino acid residues may be any natural or unnatural amino acid having a side chain which is capable of having a positive charge at physiological pH. A preferred basic amino acid is arginine. Without wishing to be bound by theory, it is believed that the inclusion of positively-charged amino acids as the second and fifth amino acids in the sequence enables the moiety to bind to the DEVD region of PARP-1 as shown in FIG. 13.

Suitable X1 moieties include those described as CDK4 peptide regions in WO2009/112536.

Alternatively, X1 may be an anionic active moiety. Anionic active moieties may comprise a total of 5 to 6 amino acid residues, and preferably a total of 6 amino acid residues. The second and fifth amino acid residues may be acidic. Anionic active moieties are believed to act as competitive inhibitors of the proteases which cleave PARP, such as caspase-3.

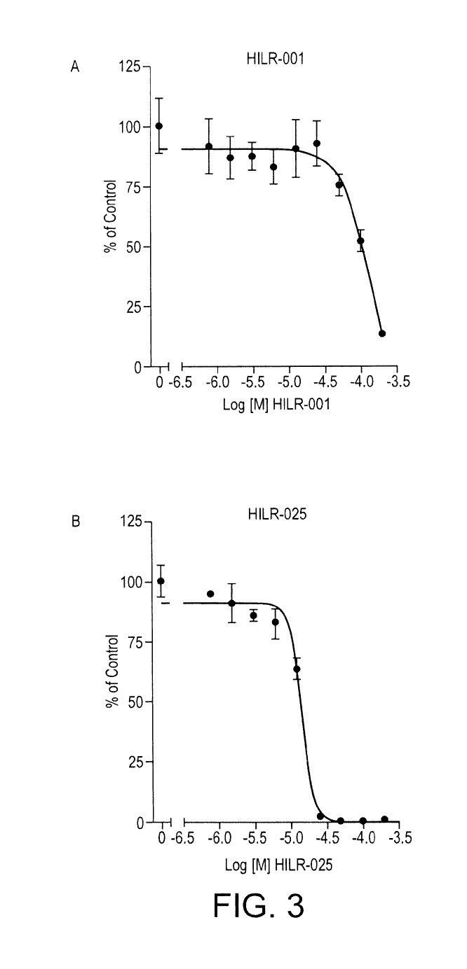

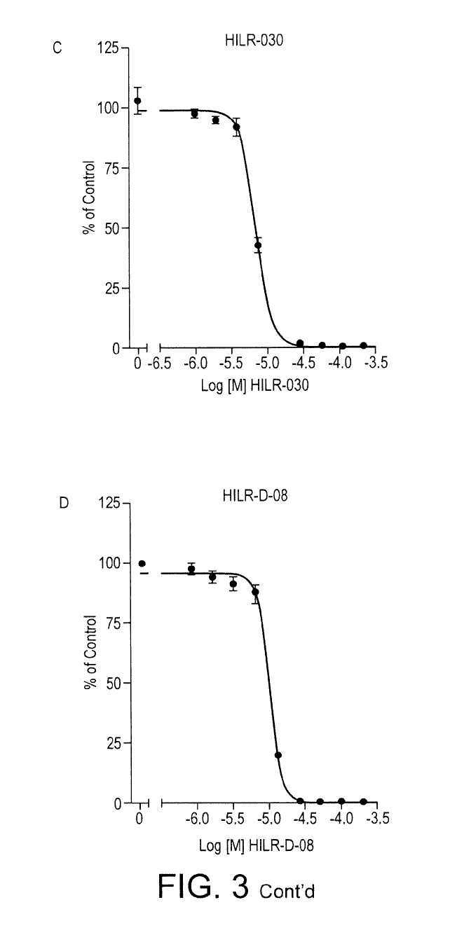

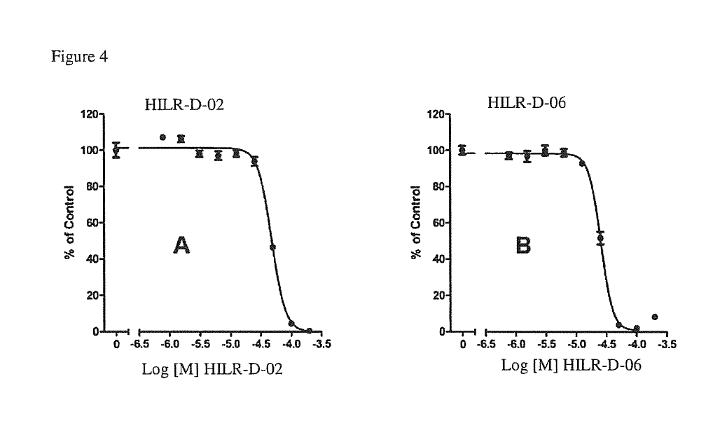

X1 may represent a peptide moiety comprising a total of 6 amino acid residues, wherein the second and fifth amino acid residues are either both basic or both acidic. A skilled artisan will be familiar with conventional assays for determining enzyme activity in the presence of an active agent. The X1 moiety will be effective in killing cancer cells. Therefore, X1 groups with suitable activity may be identified using cell viability assays. Methods measuring cell viability include the use of alamarBlue® cell viability reagent (Life Technologies, Inc.) (resazurin) with fluorescence detection. A typical experimental protocol is detailed in the Examples below. Cancer cell killing specific activity is determined by comparison of the half maximal inhibitory concentration (IC50) values for each agent (See FIGS. 3 and 4). The cyclic compound may have an IC50 of 75 μM or less, or 50 μM or less, or 30 μM or less, or 15 μM or less or 10 μM or less.

Preferably, X1 is selected from SEQ ID No. 21 (Formula 2), SEQ ID NO: 22 (Formula 3), SEQ ID NO: 23 (Formula 4) and SEQ ID NO: 24 (Formula 5):

-Pro-X5-X6-Pro-X7-Pro- SEQ ID NO: 21 (Formula 2):
wherein both X5 and X7 are amino acid residues bearing acidic side chains or wherein both X5 and X7 are amino acid residues bearing basic side chains;
wherein the amino acid residues bearing acidic side chains are each independently selected from Glu, Aza and Hca;
and
wherein X6 is selected from Gly, Ala, MeGly and (CH₂)₃;

-Pro-X8-Gly-Pro-X9-Pro- SEQ ID NO: 22 (Formula 3):
wherein X8 and X9 are each independently selected from Asp and Glu;

-Pro-Arg-Lys-Pro-Arg-Pro- SEQ ID NO: 23 (Formula 4):

-Gly-X11-Glu-Val-X12-X13- SEQ ID NO: 24 (Formula 5):
wherein X11 is selected from Asp and Glu;
wherein X12 is selected from Asp, an N-alkyl aspartic acid residue, an N-aryl aspartic acid residue, Glu, an N-alkyl glutamic acid residue and an N-aryl glutamic acid residue;
wherein X13 is selected from Gly, an N-alkyl glycine residue, and an N-aryl glycine residue;
with the proviso that if X12 is Asp, X13 is an N-alkyl glutamic acid residue or an N-aryl glutamic acid residue.

X1 moieties according to Formula 2 are particularly preferred.

In the moieties of Formula 2, X5 and X7 are preferably each independently selected from Glu and Hca. In one arrangement, X5 is Glu and X7 is Glu. In another, X5 is Glu and X7 is Hca. In a still further arrangement, X5 is Hca and X7 is Glu. In another arrangement, X5 is Hca or Aza and X7 is Hca or Aza.

In an alternative arrangement, X5 and X7 are both amino acid residues haring basic side chains. Examples of basic amino acids include Arg, Lys, and His. In this arrangement, X5 and X7 are preferably Arg. X6 is preferably a glycine residue or a sarcosine (N-methylglycine) residue. Most preferably, X6 is Gly.

Specific X1 moieties according to Formula 2 include: -Pro-Arg-Gly-Pro-Arg-Pro- (SEQ ID No: 2); -Pro-Glu-Gly-Pro-Glu-Pro- (SEQ ID No: 4); -Pro-Hca-Gly-Pro-Hca-Pro- (SEQ ID NO: 25); -Pro-Hca-MeGly-Pro-Hca-Pro- (SEQ ID NO: 26); -Pro-Aza-MeGly-Pro-Aza-Pro-(SEQ ID NO: 27); -Pro-Hca-Gly-Pro-Aza-Pro- (SEQ ID NO: 28); -Pro-Aza-Gly-Pro-Hca-Pro-(SEQ ID NO: 41); and -Pro-Aza-Gly-Pro-Aza-Pro (SEQ ID NO: 42). Of these moieties, -Pro-Arg-Gly-Pro-Arg-Pro- (SEQ ID NO: 2) and -Pro-Glu-Gly-Pro-Glu-Pro- (SEQ ID NO: 4) are preferred, and Pro-Hca-Gly-Pro-Hca-Pro (SEQ ID NO: 25) is particularly preferred.

Alternatively, the X1 moiety may be a moiety according to Formula 3 (SEQ ID NO: 22):

-Pro-X8-Gly-Pro-X9-Pro- Formula 3:

X8 and X9 are independently selected from Asp and Glu are preferably Asp.

The X1 moiety may alternatively be a moiety according to Formula 5 (SEQ ID NO: 25):

-Gly-X11-Glu-Val-X12-X13-

At least one of the amino acid residues X12 and X13 must include a chemical modification which prevents or reduces cleavage of the X12-X13 peptide bond by caspase 1. Therefore, if X12 is Asp, X13 is an N-alkyl or N-aryl glutamic acid residues. Suitable N-alkyl groups which may be present in the X12 or X13 residues include C1 to C6 linear or branched alkyl groups and C4 to C6 cycloalkyl groups. Preferably, the N-alkyl groups are C1 to C3 linear alkyl groups, most preferably methyl.

Preferably, X11 is Asp and X12 is Asp or N-methyl Asp. Most preferably, the moiety according to Formula 5 is -Gly-Asp-Glu-Val-NMeAsp-MeGly-Val- (SEQ ID NO: 29).

In a still further alternative arrangement, X1 is a moiety of Formula 6 as described in the discussion of the second aspect of the disclosure, below.

The moieties according to Formula 1 optionally comprise an X2 group. The X2 group is believed to function as a linker. The X2 group, if present, is suitably selected from Val or Ser. The X2 group is preferably present and is preferably Val. In derivatives of the moieties according to Formula 1, X2 if present may be any amino acid residue.

The sequence X3-X4-X3-X4-X3 as recited in Formula 1 represents the cassette. The cassette may improve the cell uptake of the compound and/or constrain the warhead in an optimal confirmation for bioactivity. Suitably, the cassette is amphiphilic. It is desirable for the cassette to be sufficiently hydrophilic to allow the cyclic compound to be soluble in water, while being sufficiently lipophilic to allow the uptake of the cyclic compound by a cell.

One of X3 and X4 is selected from Trp-Trp and Ar1-Ar2. The other of X3 and X4 is selected from Arg-Arg, Gpa-Gpa, Hca-Hca, and Ar3-Ar4.

Although specific arrangements of X3 and X4 are described below, it will be appreciated that alternatives to all of the described arrangements may be arrived at simply by swapping X3 and X4. For brevity, the alternatives obtainable by swapping X3 and X4 are not set out in full below. They nevertheless form part of this disclosure. By way of illustration, in particularly preferred arrangements X3 is selected from Trp-Trp and Ar1-Ar2, and X4 is selected from Arg-Arg, Gpa-Gpa, and Hca-Hca. It is also possible for X4 to be Ar3-Ar4. In the swapped configurations complimentary to these arrangements, X3 is instead selected from Arg-Arg, Gpa-Gpa, Hca-Hca and Ar3-Ar4; and X4 is instead selected from Trp-Trp and Ar1-Ar2.

Ar1, Ar2, Ar3 and Ar4 each represent unnatural amino acid residues bearing an aryl side chain. Each aryl side chain may be independently selected from an optionally substituted napthyl group, an optionally substituted 1,2-dihydronapthyl group, and an optionally substituted 1,2,3,4-tetrahydronapthyl group. The preferred aryl group is an optionally-substituted napthyl group. One or more aryl side chain may optionally be configured to act as labelling moieties.

Ar1, Ar2, Ar3 and Ar4 may be selected from amino acid residues of 3-amino-3-aryl-propionic acid or 2-amino-2-aryl acetic acid. Alternative amino acid residues include glutamic acid derivatives having the following structure:

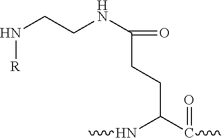

wherein R is selected from an optionally substituted napthyl group, an optionally substituted 1,2-dihydronapthyl group, and an optionally substituted 1,2,3,4-tetrahydronapthyl group.

Generally, if the aryl groups comprise substituents, lipophilic substituents are preferred. Examples of lipophilic substituents include alkyl groups, alkene groups, and alkyne groups.

Such groups may for example comprise a total of 1 to 5 carbon atoms, and may be linear or branched. Polar or charged substituents are tolerated but may reduce the rate of uptake of the compound by a cell. Typically, polar or charged side chains are included only in arrangements where the aryl side chain is to act as a labelling moiety.

In arrangements where the compound comprises a labelling moiety, substituents if present may be configured such that the aryl side chain acts as a labelling moiety. In this arrangement the aryl side chain is preferably configured to act a fluorescent label. For example, Ar1 and/or Ar2 may be Eda residues. Eda residues are fluorescent.

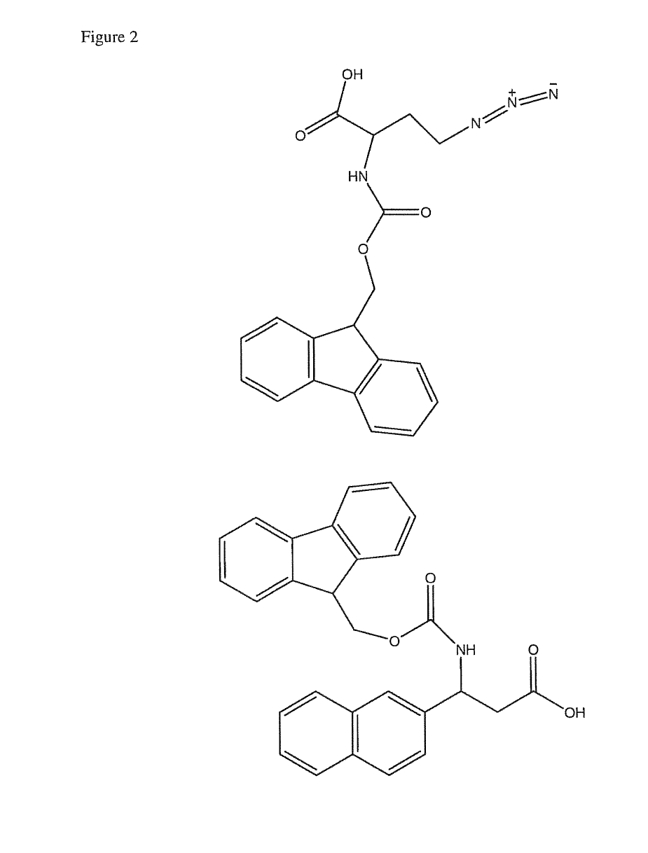

Preferably, Ar1 and Ar2 are amino acid residues of 3-amino-3-aryl-propionic acid. Most preferably, Ar1 and Ar2 are amino acid residues of 3-amino-3-(-2-napthyl)-propionic acid (“Nap”). The structure of a commercially available Fmoc-protected unnatural amino acid having a napthyl side chain is shown in FIG. 2.

In one arrangement, X3 is Ar1-Ar2 and X4 is Ar3-Ar4, Ar1 and Ar2 are each Eda, and Ar3 and Ar4 are each Nap.

In one arrangement, X3 is Trp-Trp and X4 is selected from Arg-Arg, Gpa-Gpa, and Hca-Hca. In this arrangement, X4 is preferably Arg-Arg or Gpa-Gpa.

In a particularly preferred arrangement, X3 is Nap-Nap and X4 is Arg-Arg.

Suitably, the cyclic compound comprising the moiety of Formula 1 comprises a total of less than or equal to acid 100 amino acid residues, preferably less than or equal to 50 amino acid residues, and more preferably less than or equal to 25 amino acid residues. Even more preferably, the cyclic compound comprises a total of 16 to 18 amino acid residues. The cyclic compound may consist of cyclo-[X1-X2-X3-X4-X3-X4-X3]. Examples of preferred compounds are as follows:

(SEQ ID NO: 15)
cyclo-[Pro-Arg-Gly-Pro-Arg-Pro-Val-Trp-Trp-Arg-
Arg-Trp-Trp-Arg-Arg-Trp-Trp];
(SEQ ID NO: 16)
cyclo-[Pro-Arg-Gly-Pro-Arg-Pro-Val-Trp-Trp-Gpa-
Gpa-Trp-Trp-Gpa-Gpa-Trp-Trp];
(SEQ ID NO: 19)
cyclo-[Pro-Glu-Gly-Pro-Glu-Pro-Val-Trp-Trp-Arg-
Arg-Trp-Trp-Arg-Arg-Trp-Trp];
(SEQ ID NO: 20)
cyclo-[Pro-Hca-Gly-Pro-Hca-Pro-Val-Trp-Trp-Arg-
Arg-Trp-Trp-Arg-Arg-Trp-Trp];
(SEQ ID NO: 30)
cyclo-[Pro-Hca-Gly-Pro-Hca-Pro-Val-Trp-Trp-Gpa-
Gpa-Trp-Trp-Gpa-Gpa-Trp-Trp];
(SEQ ID NO: 31)
cyclo-[Pro-Hca-Gly-Pro-Hca-Pro-Ser-Nap-Nap-Arg-
Arg-Nap-Nap-Arg-Arg-Nap-Nap];
(SEQ ID NO: 32)
cyclo-[Pro-Arg-Gly-Pro-Arg-Pro-Val-Eda-Eda-Arg-
Arg-Eda-Eda-Arg-Arg-Eda-Eda];
(SEQ ID NO: 33)
cyclo-[Pro-Hca-Gly-Pro-Aza-Pro-Val-Trp-Trp-Arg-
Arg-Trp-Trp-Arg-Arg-Trp-Trp];
(SEQ ID NO: 34)
cyclo-[Pro-Hca-Gly-Pro-Hca-Pro-Val-Nap-Nap-Hca-
Hca-Nap-Nap-Hca-Hca-Nap-Nap];
(SEQ ID NO: 35)
cyclo-[Pro-Hca-Gly-Pro-Aza-Pro-Val-Nap-Nap-Hca-
Hca-Nap-Nap-Hca-Hca-Nap-Nap];
(SEQ ID NO: 36)
cyclo-[Pro-Aza-MeGly-Pro-Aza-Pro-Val-Nap-Nap-
Hca-Hca-Nap-Nap-Hca-Hca-Nap-Nap];
and
(SEQ ID NO: 40)
cyclo-[Gly-Asp-Glu-Val-MeAsp-MeGly-Val-Trp-Trp-
Arg-Arg-Trp-Trp-Arg-Arg-Trp-Trp].

Additional examples of preferred compounds are as follows:

(SEQ ID NO: 43)
cyclo-[Pro-Hca-Gly-Pro-Hca-Pro-Val-Arg-Arg-Nap-
Nap-Arg-Arg-Nap-Nap-Arg-Arg];
(SEQ ID NO: 44)
cyclo-[Pro-Aza-Gly-Pro-Aza-Pro-Ser-Arg-Arg-Nap-
Nap-Arg-Arg-Nap-Nap-Arg-Arg];
(SEQ ID NO: 45)
cyclo-[Pro-Aza-Gly-Pro-Aza-Pro-Ser-Gpa-Gpa-Nap-
Nap-Gpa-Gpa-Nap-Nap-Gpa-Gpa];
(SEQ ID NO: 46)
cyclo-[Pro-Hca-Gly-Pro-Hca-Pro-Ser-Eda-Eda-Nap-
Nap-Eda-Eda-Nap-Nap-Eda-Eda];
(SEQ ID NO: 47)
cyclo-[Pro-Aza-Gly-Pro-Aza-Pro-Ser-Eda-Eda-Nap-
Nap-Eda-Eda-Nap-Nap-Eda-Eda];
and
(SEQ ID NO: 48)
cyclo-[Pro-Arg-Gly-Pro-Arg-Pro-Ser-Eda-Eda-Nap-
Nap-Eda-Eda-Nap-Nap-Eda-Eda].

Also contemplated herein are compounds which are salts, derivatives, prodrugs or mimetics of the cyclic compounds defined herein.

When the cyclic compounds comprise an ionisable functional group, the compound may be provided in the form of a salt with an appropriate counterion. The counterion is preferably a pharmaceutically-acceptable counterion. One of skill in the art will be familiar with the preparation of salts.

If the compound comprises acidic functional groups, the counterion may be an alkali metal or alkaline earth metal ion, for example. A preferred counterion for acidic compounds is sodium.

If the cyclic compound comprises basic amino acid residues, a salt may be formed with a strong acid or a weak acid. For example, the compound could be provided as a hydrochloride salt, a hydrogen citrate salt, a hydrogen tosylate salt, or the like.

Derivatives of the compounds described herein are also contemplated.

A derivative is a compound having substantially similar structure and function to the compounds defined herein, but which deviates slightly from the defined structures, for example by including one or more protecting groups and/or up to two additions, omissions, or substitutions of amino acid residues.

As used herein, the term “derivative” encompasses compounds in which the amino acid side-chains present in the compound are provided as protected amino acid side chains. One of skill in the art will be familiar with the use of protecting groups.

Derivatives further encompass compounds having greater than 87%, 88%, 93%, 94%, or 99% sequence homology to the compounds defined herein. To form a derivative of a compound defined herein, one amino acid residue may be omitted, replaced, or inserted. Two amino acid residues may be omitted, replaced, or inserted.

Some compounds defined herein comprise amino acid residues having N-alkyl and/or N-aryl groups. Derivatives encompass compounds in which one or more N-alkyl or N-aryl groups has been modified. An N-aryl or N-alkyl group may be modified to include a heteroatom (e.g. by replacing an alkyl —CH₂— with an ether oxygen) or a substituent such as a halogen or hydroxyl group (e.g. by replacing an alkyl —CH₂— with —CHCl—).

Also contemplated herein are pro-drugs of the cyclic compounds. A pro-drug is a compound which is metabolised in vivo to produce the cyclic compound. One of skill in the art will be familiar with the preparation of pro-drugs.

Also contemplated herein are peptide mimetics. A peptide mimetic is an organic compound having similar geometry and polarity to the compounds defined herein, and which has a substantially similar function. A mimetic may be a compound in which the NH groups of one or more peptide links are replaced by CH₂ groups. A mimetic may be a compound in which one or more amino acid residues is replaced by an aryl group, such as a napthyl group. Generally, peptide mimetics may be thought of as derivatives of peptides in which one or more of the amino acid residues is replaced by an optionally-substituted napthyl group, an optionally substituted 1,2-dihydronapthyl group, an optionally-substituted 1,2,3,4-tetrahydronapthyl group bearing a substituent, or an optionally-substituted propyl group. Substituents, if present, are typically selected from those groups which form the side-chains of any of the 23 proteinogenic amino acids. Suitably, 50% of the amino acid residues or fewer are replaced by these groups, and preferably, 25% or fewer.

Examples of mimetics of the X1 group are provided in FIG. 13.

In a second aspect, the present disclosure provides a compound capable of modulating the activity of poly(ADP-ribose) polymerase 1, which compound comprises a moiety according to Formula 6:

-Pro-X14-X15-Pro-X16-Pro- Formula 6:

wherein X14 and X16 are each independently selected from an amino acid residue bearing a side-chain, a napthyl group bearing a substituent, a 1,2-dihydronapthyl group being a substituent, a 1,2,3,4-tetrahydronapthyl group bearing a substituent, and a propyl group bearing a substituent, wherein each side-chain or substituent comprises an acidic functional group; and
wherein X15 is selected from Gly, Ala, MeGly, and (CH₂)₃.

The moiety according to Formula 6 is an anionic warhead moiety, that is, the moiety of Formula 6 may modulate the activity of poly(ADP-ribose) polymerase 1. Without wishing to be bound by theory, it is believed that anionic warhead moieties act as competitive inhibitors of proteases which cleave PARP. Surprisingly, it has been found that anionic warhead groups display useful activity.

Preferably, X14, X15 and X16 are each amino acid residues. In this arrangement, Formula 6 represents SEQ ID NO: 37. X14 and X16 may, for example, be independently selected from Asp, Glu and Hca. Preferably, when X15 is Gly one or more of X14 and X16 is not Glu.

One or more of X14 and X16 may comprise a sulfonic acid group. Compounds comprising sulfonic acid groups have been found to be particularly effective. An example of an amino acid residue comprising a sulfonic acid group is Hca.

Alternatively, the sulfonic acid group may be present as a substituent on a napthyl group, 1,2-dihydronapthyl group, 1,2,3,4-tetrahydronapthyl group, or a propyl group.

In the arrangements where the moiety of Formula 6 comprises in the main chain one or more of a napthyl group bearing a substituent, a 1,2-dihydronapthyl group being a substituent, a 1,2,3,4-tetrahydronapthyl group bearing a substituent, and a propyl group bearing a substituent, the resulting compound may be considered a peptide mimetic.

The compound may be a cyclic compound comprising a total of 16 to 18 units, wherein each unit is an amino acid residue, an optionally substituted napthyl, 1,2-dihydronapthyl or 1,2,3,4-tetrahydronapthyl group, or an optionally substituted propyl group. Preferably, each of the units in the compound is an amino acid residue. Most preferably, the compound is of Formula 8:

cyclo-[X17-X2-X3-X4-X3-X4-X3] Formula 8:

Wherein X17 is the moiety according to Formula 6, and X2, X3 and X4 are as defined above.

Also provided are salts, derivatives, prodrugs and mimetics of the cyclic compounds comprising the moiety of Formula 6.

In a third aspect, the present disclosure provides pharmaceutical compositions comprising the compounds defined herein. The pharmaceutical compositions further comprise a pharmaceutical carrier, diluent or excipients. The skilled artisan will be familiar with the formulation of pharmaceutical compositions. Any appropriate carrier, diluent or excipient may be used. Combinations of carriers, diluents and excipients may be used.

The composition may be formulated for any desired method of administration, for example for oral administration or parenteral administration.

In one arrangement, the composition may comprise an excipient which is a delivery component as defined in US Patent Application Publication No. 2003/0161883.

Optionally, the pharmaceutical compositions comprise a further therapeutic agent. Preferably, the further therapeutic agent is an aerobic glycolysis inhibitor. The co-administration of the compositions of the present disclosure with an aerobic glycolysis inhibitor produces an additive or synergistic effect when used in the treatment of cancer. The preferred aerobic glycolysis inhibitor is 2-deoxyglucose (2-DOG). 2-deoxyglucose is generally well tolerated in vivo. Administering 2-deoxyglucose in combination with the compositions of the present disclosure may allow the dosage of the compounds of the present disclosure to be reduced.

Preferably, the compounds and pharmaceutical compositions of the present disclosure are for use in medicine. Preferably, the compounds and compositions are for use in a method of treating cancer, which method comprises administering to a patient the compound or composition. The method may further comprise the use of conventional methods for the treatment of cancer, such as the use of radiation therapy and/or surgery. The compounds and compositions of the invention may be formulated for administration as part of a method comprising the use of other chemotherapeutic agents.

The putative mechanism of action of the compounds of the present disclosure, discussed in more detail below, indicates that the compounds will be useful in the treatment of a wide range of cancers. It follows that the compounds may be useful for the treatment of a patient suffering from multiple cancers or metastatic cancer.

Since the compounds of the present disclosure modulate the activity of PARP-1, the compounds and compositions of the present disclosure are particularly well adapted for use in the treatment of a cancer comprising cancer cells in which PARP-1 is up-regulated relative to non-cancerous cells. Cancers in which PARP-1 may be up-regulated include breast cancer, colon cancer, endometrial cancer, oesophageal cancer, kidney cancer, lung cancer, ovarian cancer, rectal cancer, stomach cancer, thyroid cancer and testicular cancer.

The compounds and compositions of the present disclosure may be used in the treatment of a patient suffering from a cancer, wherein the cancer comprises one or more of: breast cancer, prostate cancer, colorectal cancer, bladder cancer, ovarian cancer, endometrial cancer, cervical cancer, head and neck cancer, stomach cancer, pancreatic cancer, oesophagus cancer, small cell lung cancer, non-small cell lung cancer, malignant melanoma, neuroblastoma, leukaemia, lymphoma, sarcoma or glioma. Preferably, the cancer is selected from breast cancer, colon cancer, endometrial cancer, oesophageal cancer, kidney cancer, lung cancer, ovarian cancer, rectal cancer, stomach cancer, thyroid cancer and testicular cancer.

Also provided herein is the use of the compounds defined herein to modulate the activity of PARP-1 in vitro. The use may comprise, for example, contacting a cell culture or tissue sample with a compound as defined herein. The cell culture or tissue sample may comprise immortalised human cells, optionally cancer cells. The tissue sample may be, for example, a biopsy from a patient suffering from a cancer.

In a still further aspect, the present invention provides a method of analysis, which method comprises contacting cells with a compound of the present disclosure and detecting the compound. Suitably, the compound comprises a labelling moiety.

The cells may be contacted with an additive, excipient, or co-active. This may allow the effect of additives, excipients and co-actives on, for example, the uptake of the compound by the cells to be investigated.

The method of detection may be selected as appropriate. When the compound comprises a labelling moiety, an appropriate method of detection is selected depending on the nature of that moiety. Of course, the method may comprise additional intermediate steps. The method of analysis may for example comprise steps used in conventional assays for investigating cells. In one arrangement, the method comprises a Western blot analysis.

One illustrative method for detecting the compound is fluorescence detection. In this arrangement, the compound suitably comprises a labelling moiety which is fluorescent. Tryptophan residues are also capable of fluorescence.

Typically, the method of analysis is performed in vitro. The sample may be a cell culture. The sample may be a biopsy obtained from a patient, or derived from such a biopsy. In the arrangements where the cells are obtained from a patient, the analysis may have diagnostic applications.

Without being bound by theory, the following mechanism is suggested to explain the mode of action of the compounds of the present disclosure.

### PRGPRP Function in Normal Cells:

Cdk4 with its cyclin D partners initiates the molecular processes which begin cell division by phosphorylating the retinoblastoma protein (pRb) and associated pRb family members (Harbour et al. Cell (1999); 98: 859-869), leading to the release of E2F-1 and associated proteins involved in the induction of the relevant enzymes for DNA synthesis (Classon and Harlow; Nature Reviews Cancer (2002) 2: 910-917). In addition to promoting cellular proliferation, however, E2F can induce apoptosis (Nevins et al., Hum Mol Genet. (2001); 10:699-703).

It is proposed that in normal diploid cells the PRGPRP region of Cdk4 (SEQ ID NO: 2) guards against apoptosis by E2F-1 when the kinase region of Cdk4 phosphorylates the Rb protein and related family members. Protection against apoptosis is achieved by PRGPRP (SEQ ID NO: 2) binding to the DEVD region of PARP (SEQ ID NO: 1) and thus impeding caspase-3 (and others) binding at that site so that PARP is not cleaved. Cleavage of PARP-1 by caspases is considered to be a hallmark of apoptosis [Kaufmann S H, et al: Specific proteolytic cleavage of poly(ADP-ribose) polymerase: an early marker of chemotherapy-induced apoptosis. Cancer Res 1993, 53:3976-3985. Tewari M, et al. Yama/CPP32 beta, a mammalian homolog of CED-3, is a CnnA inhibitable protease that cleaves the death substrate poly(ADP-ribose) polymerase. Cell 1995, 81:801-809]. Thus by “applying a brake” to PARP-cleavage, the PRGPRP domain of CDK4 mediates against excessive apoptosis.

In normal cells there is little to no DNA damage so there will be minimal Poly(ADP-ribosylation) and the PRGPRP-protected uncleaved PARP will not deplete NAD+ which will remain at high enough levels.

### PRGPRP Function in Early Multistage Carcinogenesis:

Several reports indicate that Cdk4, in contrast to Cdk2 or Cdk6, appears to be the sole cyclin-dependent kinase whose functioning presence is mandatory for successful tumorogenesis (Warenius et al., Molecular Cancer (2011); 10: 72-88.).

In summary: Cdk4 gene knockout in mice completely abrogates chemically induced epidermal carcinogenesis (Rodriguez-Puebla et al., 2002; Am J Pathol (2002); 161: 405-411.), without effect on normal skin keratinocyte proliferation, despite the continuing presence of Cdk2 and Cdk6. Additionally, ablation of CDK4 (Miliani de Marval et al.; Mol Cell Biol. (2004); 24: 7538-7547) but not of CDK2 (Macias et al. 2007; Cancer Res 2007, 67:9713-9720) inhibits myc-mediated oral tumorigenesis. Furthermore, overexpression of Cdk4 but not cyclin D1 promotes mouse skin carcinogenesis (Rodriguez-Puebla et al., 1999; Cell Growth Differ 1999, 10:467-472.), whilst elevated Cdk2 activity, despite inducing keratinocyte proliferation, is not tumorogenic (Macias et al. 2008).

Multistage carcinogenesis occurs as the result of deregulation of both cell proliferation and cell survival (Evan and Vousden 2001; Nature (2001); 411: 342-348). Activating mutations occur in genes promoting cell division and inactivating mutations occur in tumour suppressor genes. However, mutations that can activate the pathways leading to deregulation of E2F factors and promote increased cellular proliferation can also promote apoptosis (Quin et al. 1994; Proc. Natl Acad. Sci. USA (1994); 91: 10918-10922, Shan et al. 1994; Mol. Cell. Biol (1994); 14: 8166-8173). For carcinogenesis to progress successfully, cells must be able to maximise proliferation whilst avoiding apoptosis (Lowe and Lin 2000; Carcinogenesis (2000); 21: 485-495).

An explanation for the above findings could be that during carcinogenesis there is an increased likelihood of apoptosis as well as cellular proliferation. By binding to DEVD and preventing PARP cleavage, the PRGPRP motif inhibits apoptosis allowing tumours to form. In the absence of PRGPRP increased apoptosis will prevent tumour formation. Early in carcinogenesis DNA damage is minimal, cell division is not unrestrained and the cell is not operating under aerobic glycolysis, so preventing PARP cleavage will be unlikely to cause necrosis.

The observation that the presence of Cdk4 appears to be mandatory for successful carcinogenesis can therefore be explained, not by reference to the kinase activity of Cdk4, but rather by the activity of the externalised loop containing the PRGPRP motif, which binds to the DEVD region of PARP minimises apoptosis and allows increased cellular proliferation to progress.

In the absence of Cdk4 and its PRGPRP (SEQ ID NO: 2) site the carcinogenic process is likely to end in apoptosis rather than cell immortalisation.

### The Effect of the PRGPRP Region of CDK4 in Fully Developed Cancer Cells:

It has become increasingly apparent over the past decade that the DNA of established cancer cells is massively damaged (Warenius; Anticancer Res. (2002); 22:2651-2656). This high level of DNA damage is not a feature of early carcinogenesis but has been observed across a wide range of clinical cancers (Sjöblom et al., Science (2006): 314: 268-274; Greenman et al., 2007; Jones et al., Science (2008); 321: 1801-1806; Gerlinger et al., N Engl J Med (2012); 366: 883-892). Cell lines used in HilRos research have been derived from similar advanced cancers and will thus also exhibit similar massive DNA damage.

Significant DNA damage would be expected to stimulate PARP to carry out poly(ADP-ribosylation) at multiple sites, using up the available NAD+. Upregulation of PARP-1 has been described in many tumour types including breast, colon, endometrial, oesophagus, kidney, lung, ovary, skin, rectal stomach, thyroid and testisticular cancer (Ossovskaya et al. Genes and Cancer (2010); 1: 812-821). The cell also responds to DNA damage by activating the apoptotic pathway which involves caspase cleavage of PARP at the DEVD site thus inactivating poly(ADP-ribosylation) and allowing sufficient NAD+ to generate the ATP that is necessary for apoptosis. The survival of such advanced cancer cells is thus dependent on a balance between a tendency towards apoptotic death or necrotic death.

In addition the unrestrained division of cancer cells, in contrast to normal cells, requires increased energy for the synthesis of new cellular macromolecules and the accomplishment of mitosis.

Finally the Warburg effect in cancer cells makes them much more dependent on aerobic glycolysis (which may be increased as much as 200-fold) than on mitochondrial ATP generation.

By inhibiting PARP cleavage, compounds of the present disclosure put stress on the cellular energy supplies. However, PARP agonists (and caspase inhibitors) do not cause the cancer cell necrosis seen with the present compounds. For necrosis to occur a further stress is needed. Thus peptides of the present disclosure are likely to have an additional target to PARP such as lactate dehydrogenase (LDH), which is involved in the aerobic glycolysis characteristic of cancer cells.

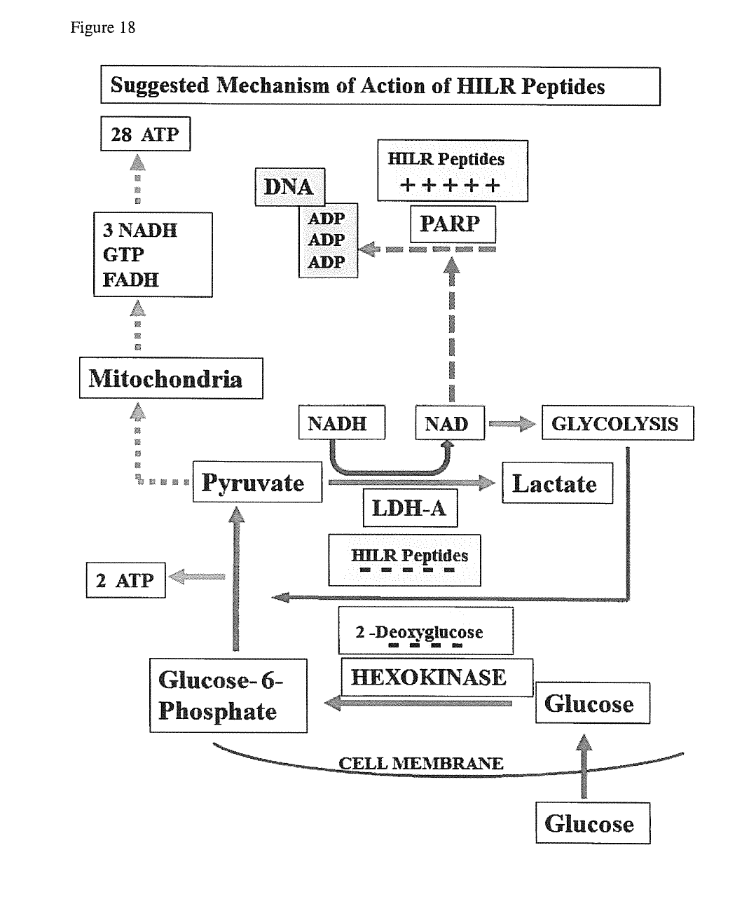

In cancer cells the switch to aerobic glycolysis makes its energy systems very dependent on the supply of NAD produced by the activity of LDH [see FIG. 18]. In this situation the cancer cell will be exquisitely sensitive to the competing demand of upregulated, active PARP for NAD to be used in poly-ADP-ribosylation. A compound whose action is like that described here for HILR cyclic peptides will be likely to be selectively toxic to cancer cells by agonizing PARP and increasing its NAD utilisation at the same time as inhibiting LDH and lowering the availability of NAD, resulting in insufficient NAD for the glycolytic, Embden-Meyerhof pathway from glucose-6 phosphate to pyruvate.

Without being bound by theory it is suggested that the peptides of the present disclosure may kill cancer cells by attacking two of their global weaknesses: the need to repair massive DNA damage and the switch to aerobic glycolysis.

### EXAMPLES

The present invention will now be described in further detail with reference to the following illustrative Examples.

### Example 1: Improved Specific Activity

Three cyclic peptides (HILR-001 (SEQ ID NO: 13), HILR-025 (SEQ ID NO: 15) and HILR-030 (SEQ ID NO: 16)) were prepared to >95% purity using a conventional automated peptide synthesis technique. HILR-001 (SEQ ID NO: 13) is a comparative compound produced in accordance with Warenius et al, Molecular Cancer (2011); 10:72-88. HILR-025 (SEQ ID NO: 15) and HILR-030 (SEQ ID NO: 16) are cyclic compounds comprising (Trp-Trp-Arg-Arg) or (Trp-Trp-Gpa-Gpa) repeats. The activity of the compounds was tested as follows:

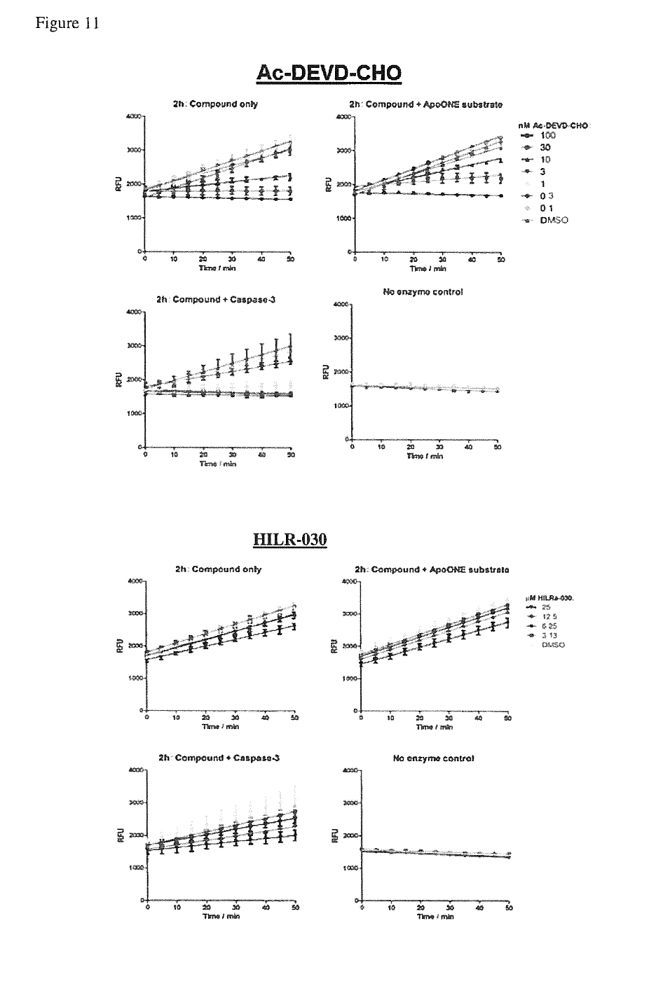

1) NCI-H460 cells were grown in Ham's F12 media supplemented with 10% FBS.
2) Cells were harvested and seeded into 96-well plates at 500 cells/well.
3) Compounds were made up from stock solutions and added directly to cells in doubling dilutions starting at 200 μM. Final DMSO concentration was 0.2%.
4) Cells were grown with compound for 96 hours at 37° C. 5% CO₂ in a humidified atmosphere.
5) A resazurin dye composition (AlamarBlue® cell viability reagent (Life Technologies, Inc.)) 10% (v/v) was then added and incubated for a further 4 hours, and fluorescent product detected using the BMG FLUOstar plate reader.
6) Media only background readings were subtracted before data were analysed using a 4-parameter logistic equation in GraphPad Prism. Results are shown in FIG. 11. The IC₅₀ of HILR-30 was determined as 6 μM.

As shown in FIG. 3, inserting the new “backbone” sequence WWRRWWRRWW (SEQ ID NO: 17) into cyclic HILR-025 along with PRGPRP (SEQ ID NO: 2) increased the specific activity compared to THR54 (HILR-001), lowering the IC₅₀ dose from 98 μM to 15 μM. Further modification to make the “backbone” more lipophilic by the substitution of guanidino-phenylalanines for arginines, yielding HILR-030, further improved the specific activity to give an IC₅₀ of 6.0 μM.

Oligomeric linear sequences comprised of arginine and tryptophan have been described as previously having successful cellular uptake properties. VIZ: RRWRRWWRRWWRRWRR (SEQ ID NO: 38) [Derossi et al. Trends in Cell Biol (1998) 8:84-87]. Cyclic arginine/tryptophan peptides as a means of enhancing cell uptake of passenger peptides, have also been described: [Cyc-(WRWRWRWR) (SEQ ID NO: 39) Shirazi et al. Mol Pharmaceutics (2013) 10:2008-2020].

However, it was not clear from the literature what sequences of arginines and tryptophans would be most effective for improving cell uptake. Whilst arginine dimers alternating with monomeric or dimeric tryptophans were described by Derossi et al. (above) in linear cell-internalising peptides, the cyclic (WR)₄ peptides described by Sherazi et al. alternated single arginines and tryptophans. There were no a priori or apparent experimental reasons why cyclic peptides with (WWRR)_{X} sequences in the “backbone” should be any more active than those with ALKL sequences.

Furthermore, the binding of the PRGPRP “warhead” (SEQ ID NO: 2) to the DEVD region of caspase-1 is dependent upon the positioning of the arginine residues, as shown in FIG. 13. It was originally believed that the presence of arginine residues in the backbone would complete or interfere with the binding of the PRGPRP warhead (SEQ ID NO: 2) to its biological target. Surprisingly, this is not the case.

### Example 2: PARP-Dependent Cytotoxicity

The present inventor hypothesized that modulation of PARP activity by a PRGPRP cyclic peptide might be, at least in part, responsible for the drop in ATP and subsequent necrosis in a human non-small cell lung cancer. HILRa cyclic peptides might thus be PARP-dependent. If so, it was postulated that this should be reversed by a PARP inhibitor such as Olaparib.

In this situation, Olaparib would diminish/prevent cell death induced by a HILRa cyclic peptide.

A study was thus carried out to examine the effect on ATP levels and cell death of NCI-H460 human non-small cell lung cancer cells exposed for 72 hours and 96 hours respectively to HILR-001 [cyc-(Pro-Arg-Gly-Pro-Arg-Pro-Val-Ala-Lue-Lys-Leu-Ala-Leu-Lys-Leu-Ala-Leu] (SEQ ID NO: 13) (Polypeptide Laboratories, France, SAS, 7 Rue de Boulogne, 67100, Strasbourg, France)] alone or co-incubated with Olaparib.

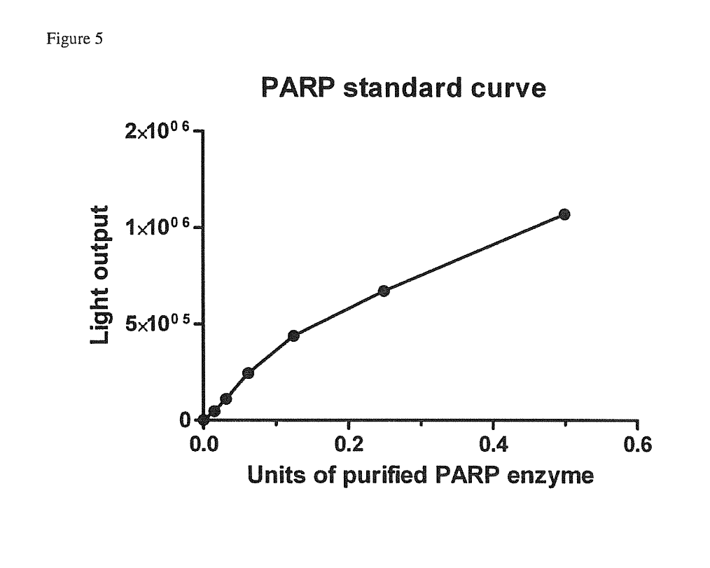

An in vitro PARP standard curve was initially produced [FIG. 5].

### Protocol:

1) NCI-H460 cells were grown in Ham's F12 media supplemented with 10% FBS.
2) Cells were harvested and seeded into 10 cm dishes at 1×10⁶ cells per dish.
3) Olaparib was prepared from stock solutions and added directly to cells to give the final concentrations indicated on the graph. DMSO content was kept constant at a concentration of 0.1%.
4) Cells were incubated with Olaparib or vehicle control at 37° C., 5% CO2 for 4 hours, 24 hours, 48 hours or 96 hours.
5) Cells were harvested at the different time points and cell pellets stored at −80° C. until the time course was complete.
6) Cell pellets were thawed and lysed in 50 μl PARP lysis buffer.
7) Protein concentrations in the samples were quantified by a BCA assay.
8) 40 μg of sample was then assayed in duplicate using the Universal Chemiluminescent PARP Assay Kit with Histone-Coated Strip Wells from Trevigen (Cat #4676-096-K), following manufacturer's instructions for PARP Activity in Cell and Tissue Extracts.
9) The 4 test concentrations of Olaparib and 2 concentrations of 3-aminobenzamide were assayed in duplicate in an in vitro assay using the above mentioned kit, following manufacturer's instructions for the PARP Inhibitor Assay Protocol.
10) Luminescent product was detected using the BMG FLUOstar plate reader.

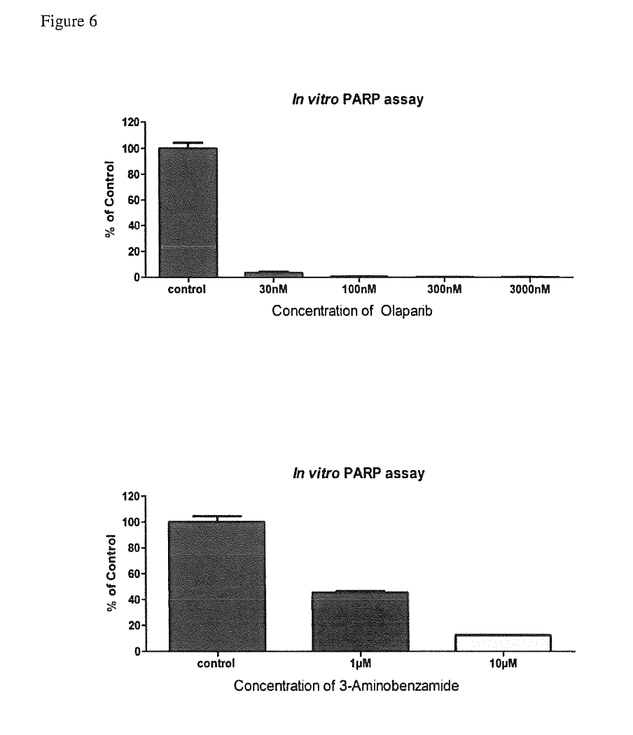

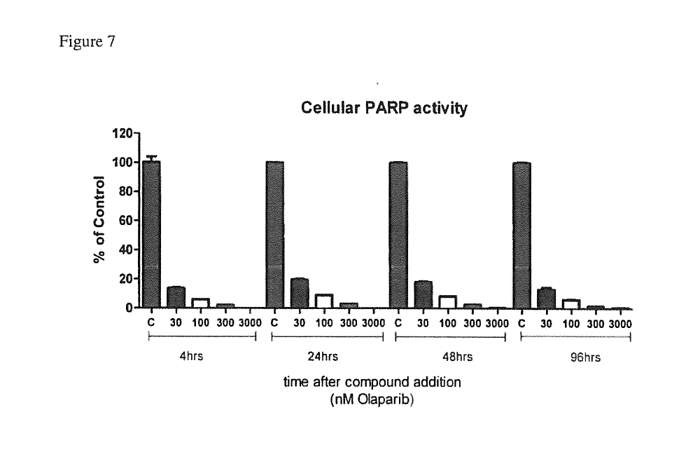

The minimal concentration of Olaparib required to produce more than 90% inhibition of PARP was compared to 3-aminobenzamide [FIG. 6] and a time course for PARP inhibition by Olaparib was plotted [FIG. 7].

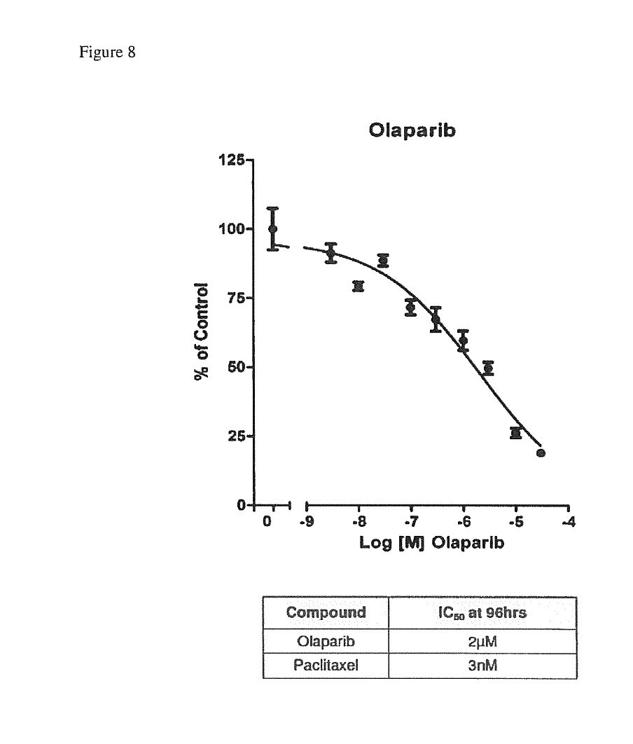

The in vitro cytotoxicity of Olaparib itself on NCI-H460 human non-small cell cancer was then tested [FIG. 8].

### Protocol:

1) NCI-H460 cells were grown in Ham's F12 media supplemented with 10% FBS.
2) Cells were harvested and seeded into 96-well plates at 500 cells/well.
3) Olaparib was made up from stock solutions and added directly to cells in semi-log dilutions starting at 30 μM. Final DMSO concentration was 0.3%.
4) Cells were grown with compound for 96 hours at 37° C. 5% CO2 in a humidified atmosphere.
5) AlamarBlue® cell viability reagent (Life Technologies, Inc.) 10% (v/v) was then added and incubated for a further 4 hours, and fluorescent product detected using the BMG FLUOstar plate reader.
6) Data were analysed using a 4-parameter logistic equation in GraphPad Prism.

A dose of 30 nM Olaparib was found to be non-toxic to NCI-H460 cells and to exhibit greater than 80% inhibition of cellular PARP activity. This dose of Olaparib was chosen for co-incubation with HILR-001 assay for 96 hours.

Four concentrations of Olaparib were tested and a dose-dependent decrease in cellular PARP activity was observed at all time-points. The 4 test concentrations of Olaparib and 2 concentrations of the control compound 3-aminobenzamide were tested in an in vitro assay using purified PARP enzyme. This assay was run in parallel to the cellular PARP assay to act as a positive control.

Effect of olaparib on ATP depletion and necrosis in NCI-H460 mediated by HILR-030:

Four concentrations of HILR-001 were tested in the presence or absence of 30 nM Olaparib; At each time point cell viability was measured by two assay readouts, alamarBlue® and CellTiter-Glo. Conversion of alamarBlue® to a fluorescent product serves as a readout of the metabolic activity of cells, whereas CellTiter-Glo is based on quantification of the ATP present.

### Protocol:

1) NCI-H460 cells were grown in Ham's F12 media supplemented with 10% FBS.
2) Cells were harvested and seeded into 96-well plates at 500 cells/well.
3) HILR-001 was made up from a 10 mM stock solution and added directly to cells in doubling dilutions starting at 200 μM. Olaparib was made up from a 10 mM stock solution and added directly to cells at 30 nM. The total final DMSO concentration was 0.25%.
4) Cells were grown with compound for 24, 48, 72 or 96 hours at 37° C. 5% CO2 in a humidified atmosphere.
5) AlamarBlue® 10% (v/v) was then added and incubated for a further 4 hours, and fluorescent product detected using the BMG FLUOstar plate reader.
6) On duplicate plates the media was removed from the cells, CellTiter-Glo was diluted in PBS (1:10) and 100 μl added to the cells.
7) Plates were mixed on an orbital shaker for 2 minutes and incubated for a further 10 minutes at room temperature. Luminescent signal was then measured using the BMG FLUOstar plate reader.

When HILR-001 was tested as a single agent, a dose dependent decrease in metabolic activity (alamarBlue®) was observed. This was particularly evident at the later time points and was consistent with previously published results (Warenius et al. Molecular Cancer (2011); 10:72-88).

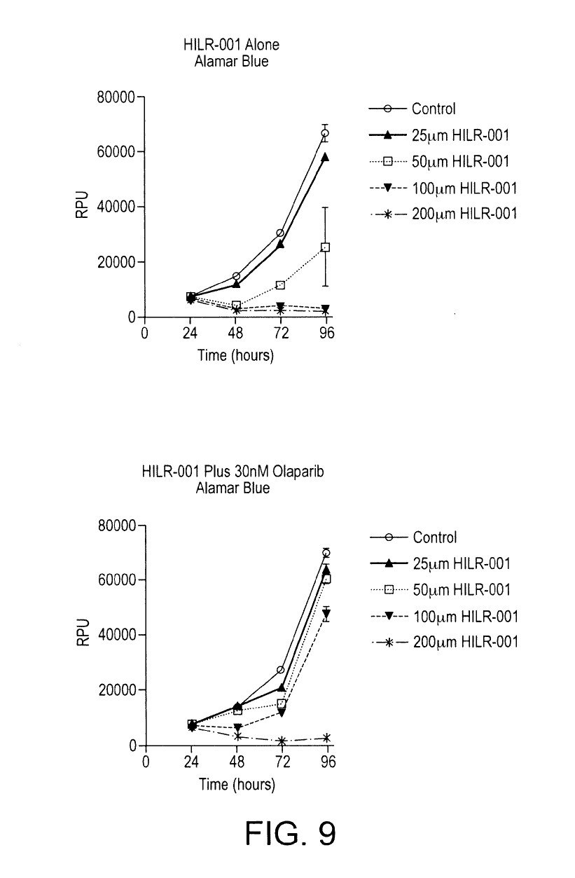

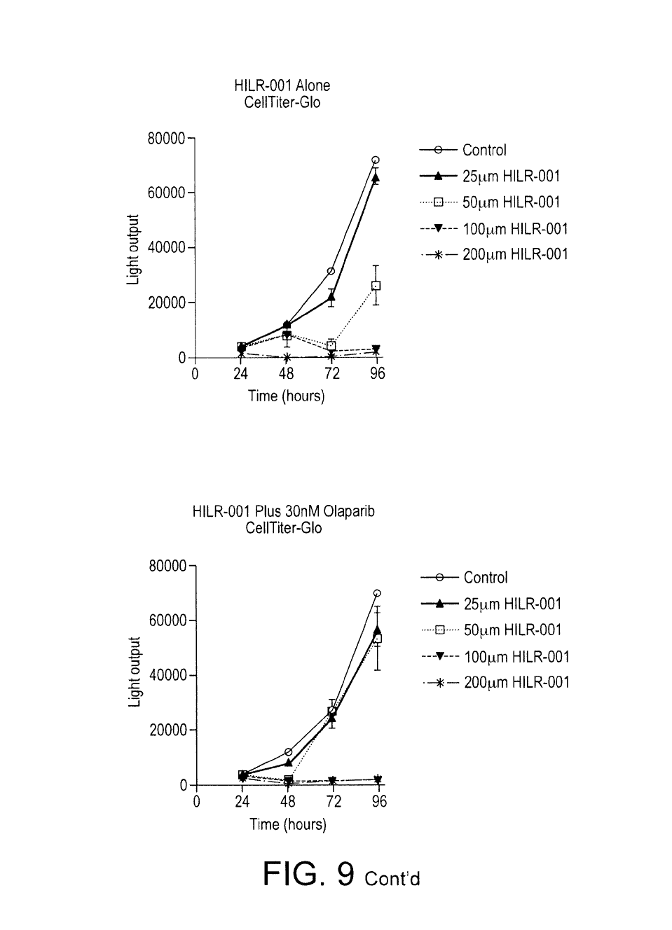

30 nM Olaparib partially restored ATP levels (Cell Titre Glo) and reversed 50 μM HILR-001-mediated cell death (alamarBlue®) [FIG. 9], demonstrating that its activity is PARP-dependent at this dose level. At higher doses of HILR-001 (100 μM and 200 μM), Olaparib did not affect ATP levels or cancer cell death, indicating that the cancerocidal action of HILR-001 is likely to be only partially explained by a mechanism involving its effect on PARP function.

The above experiments demonstrate the surprising finding that PARP activity plays a significant role in the mechanism by which PRGPRP peptides cause cancer cell necrosis and this activity can be partially reversed by a specific PARP inhibitor. The interaction of a PRGPRP peptide with PARP is thus a necessary, though not sufficient requirement for cancer cell necrosis.

### Example 3: Competitive Inhibition of DEVD

PARP activity is controlled by whether or not there has been cleavage at the DEVD site. Cleaved PARP is inactivated with regard to its poly(ADP-ribose) phosphorylation activity. A poly(ADP-ribose) phosphorylation inhibitor such as olaparib would not be expected to have any effect on cleaved PARP. Thus it is likely that PRGPRP (SEQ ID NO: 2) acts on intact PARP which will have intact DEVD region. Moreover it is proposed that the activity of HILR-001 can be explained by PRGPRP (SEQ ID NO: 2) binding to the DEVD region of PARP and thus protecting this region from caspase binding and proteolytic cleavage.

Without taking into account secondary and tertiary conformational orientation of regions within peptides in general, it is notable that the linear arrangement of aspartic acid anions in the GDEVDG region of PARP (SEQ ID NO: 1) aligns quite closely with the cationic arginines [FIG. 13], and these arginines have been shown to be key to the anticancer effects of PRGPRP (SEQ ID NO: 2) (Warenius et al. Molecular Cancer (2011); 10:72-88)

If DEVD is a downstream target of PRGPRP (SEQ ID NO: 2) then PRGPRP-unrelated molecules, which might protect PARP cleavage at the DEVD site, might also contribute to NCI-H460 cellular cytotoxicity.

Cyclic peptides were designed which by homology to GDEVDG (SEQ ID NO: 1), might competitively bind to caspases and related molecules which cleaved PARP at the DEVD site [Gly-Asp-Glu-Val-Asp₂₁₄-Gly₂₁₅] (SEQ ID NO: 1). Cleavage takes place between Asp 214 and Gly 215 amino acids to yield two fragments; an 89- and a 24-kDa polypeptide.

A GDEVDG hexapeptide, HILR-D-01 (Cyc-[Gly-Asp-Glu-Val-NMeAsp-Sarc-Val-Trp-Trp-Arg-Arg-Trp-Trp-Arg-Arg-Trp-Trp] (SEQ ID No: 40), was thus constructed with methyl amide bonds at the cleavage site and this was inserted in place of PRGPRP (SEQ ID NO: 1) into an improved cassette earlier found to increase PRGPRP specific activity (Example 1).

HILR-D-01 showed a weak but significant dose-related cell-killing, demonstrating that blocking PARP cleavage can contribute to the induction of cancer cell necrosis [FIG. 13].

### Example 4: Caspase Inhibition

To test further whether the PARP-dependence of HILR-peptides was due to PARP activity being maintained by inhibition of PARP cleavage, an assay using the Apo-ONE Homogeneous Caspase-3/7 reagent from Promega was conducted in the presence of a range of doses of HILR-030. DEVD-CHO was used as a positive control.

The Promega kit consists of a buffer that supports caspase 3/7 enzymatic activity and the caspase-3/7 substrate rhodamine 110, bis-(N-CBZL-aspartyl-L-glutamyl-L-valyl-L-aspartic acid amide; Z-DEVD-R110) Z-DEVD-R110 exists as a pro-fluorescent substrate prior to the assay; upon sequential cleavage and removal of the DEVD peptides by caspase-3/7 activity and excitation at 499 nm, the rhodamine 110 leaving group becomes fluorescent. The amount of fluorescent product generated is reported to be proportional to the amount of caspase-3/7 cleavage that occurs in the sample. (The reagent sources were Enzo Life Sciences Cat No: BML-SE169-5000); Apo-ONE® Homogeneous Caspase-3/7 Assay (Promega Cat No: G7790); Control compound Ac-DEVD-CHO Sigma Cat No: A0835).

Using a 384-well plate format, enzymatic reactions were detectable at all plate reader gain settings used; the maximum detectable signal was exceeded at a gain setting of 1000 when 10 U enzyme was present in the reaction. At the top gain setting used, an increase in fluorescence signal over time was observable when 0.01-10 units of caspase-3 were used in the reaction. Within this range, the initial rate of reaction was directly proportional to the total amount of enzyme present in the reaction. 0.3 U, 0.1 U and 0.03 U enzyme were taken forward to the next phase of optimisation using a plate reader gain setting of 1000.

Optimal recombinant human caspase 3 enzyme activity was determined by titration, demonstrating linearity of initial recombinant enzyme kinetics between enzyme doses of 0.03-0.30 units. Within this range, the initial rate of reaction was directly proportional to the total amount of enzyme present in the reaction. A DMSO tolerance assay was also carried out, demonstrating: concentrations of DMSO above 1% in the final assay appeared to reduce the initial rate of reaction; however, the rate remained linear over a 50 min period.

Within these parameters, the increase in fluorescent signal remained linear over approximately 50 min, allowing initial rates to be calculated with strong correlation coefficients, whilst remaining economical with the amount of enzyme used.

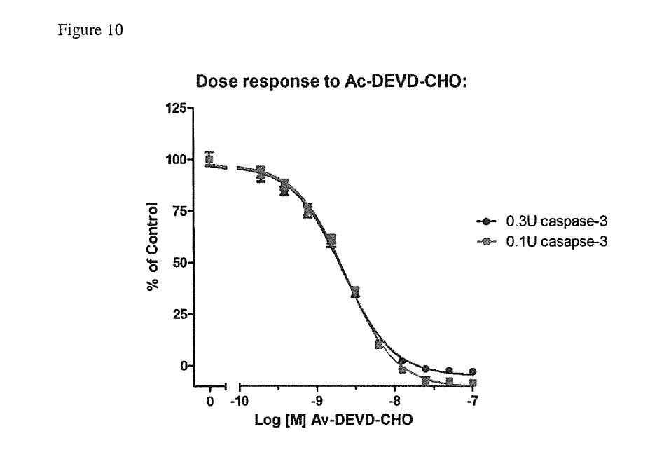

Ac-DEVD-CHO inhibited the activity of caspase-3 in a dose-dependent manner, giving rise to IC₅₀s within the expected range according to the inhibitor specification sheet [FIG. 10]. Similar inhibitor IC₅₀s were achieved when assaying against either 0.1 or 0.3 U enzyme. In all subsequent experiments, 0.1 U enzyme was used and plate reader settings were adjusted to read every 5 min for 2 h.

The DEVD-CHO control or HILR-030 were co-incubated for 2 hours with substrate or human recombinant caspase-3 according to the protocol in the table below.

| Pre-treatment | t = −2 h | t = 0 |
|---|---|---|
| No enzyme control | 5 μl compound | 25 μl ApoONE reagent |
| | 20 μl buffer | |
| 2 h compound only | 5 μl compound | 20 μl enzyme |
| | | 25 μl ApoONE reagent |
| 2 h compound | 5 μl compound | 25 μl ApoONE reagent |
| enzyme | 20 μl enzyme | |
| 2 h compound | 5 μl compound | 20 μl enzyme |
| substrate | 25 μl ApoONE reagent | |

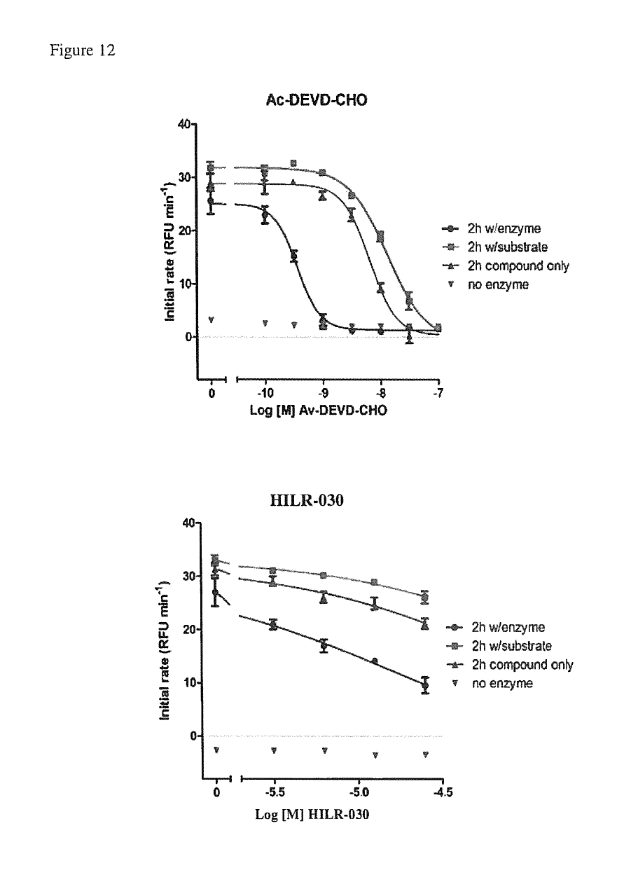

Both DEVD-CHO and HILR-030 inhibited the caspase-3 activity in a dose-dependent fashion [FIGS. 11, 12]

### Example 5: Anionic/Cationic “Warhead”

HILR-D-02 (Cyc-[Pro-Glu-Gly-Pro-Glu-Pro-Val-Trp-Trp-Arg-Arg-Trp-Trp-Arg-Arg-Trp-Trp])(SEQ ID NO: 19) was designed as a negative control for HILR-025 and tested on NCI-H460 human non-small cell cancer cells in vitro.

Surprisingly HILR-D-02 was cytotoxic towards NCI-H460 cells with an IC₅₀ of 38 μM. [FIG. 4A]. To confirm that substitution of the highly charged cationic guanidium group of arginine for an anionic group could, generally, also give rise to a cancerocidal molecule, a further HILR-025 cyclic peptide cationic analogue with sulfonic acid groups instead of guanidium groups was synthesised, by replacing the arginines of HILR-025 with homocysteic acid residues. This cyclic peptide HILR-D-06 killed NCI-H460 cells even more effectively than HILR-D-02 with an IC₅₀ of 25 μM [FIG. 4B]. It thus appears to be the case that both anionic and cationic groups in the same sites within the cyclic peptides, described here, can cause cancer cell killing in vitro.

This result is surprising because the anionic hexapeptide PEGPEP (SEQ ID NO: 4) was previously reported to be inactive [Warenius et al. Molecular Cancer (2011) 10: 72-88]. It is believed that the activity of the active anionic group was not observed in the earlier study because the duration of contact between the anionic hexapeptide and the cancer cells was not sufficient and because the concentration of PEGPEP (SEQ ID NO: 4) used was not sufficient. Often, a high dosage is required when utilising short linear peptides. It is believed that the cassette sequences included in the cyclic peptides of the present disclosure enhance the delivery of the active moiety to the cell allowing the use of lower dosages.

Without being bound by theory, it is proposed that these cyclic peptides interact by electrostatic binding to their putative target(s) and can act by both a competitive inhibition or “decoy” mechanism, thus explaining the similar effect of both anionic and cationic “warheads”.

HILR cyclic peptides likely interact with the DEVD region of PARP protecting it from cleavage and preserving PARP activity. This is necessary for the cancer cell necrosis activity of these agents but not sufficient to explain their complete mechanism of action. The proposal that these HILR peptides are partial PARP agonists is consistent with what has previously been reported for other PARP agonists (see above). HILR cyclic peptides would thus appear to have a potential dual activity a) on PARP and b) on a non-PARP effector of cellular ATP levels. Without being bound by theory, two possible candidates for this extra-PARP activity could be the enzyme lactate dehydrogenase, where arginines play an important role in binding acetyl CoA within the active enzymatic site, and hexokinase 2.

### Example 6: Effect of the Compounds of the Invention in Combination with 2-Deoxyglucose

Since the compounds according to the invention appeared to be causing cell death by necrosis as a result of NAD/ATP depletion, it was hypothesised that their activity could be potentiated by administering the compounds with a glycolysis inhibitor. The cell killing ability of HILR-025 (SEQ ID NO: 15) and HILR-D-07 sodium salt (SEQ ID NO: 30) in the presence and absence of the glycolysis inhibitor 2-deoxyglucose (2-DOG) was therefore assayed.

HILR-025 (SEQ ID NO: 15) comprises a cationic PRGPRGP (SEQ ID NO: 2) warhead, whereas HILR-D-07 (SEQ ID NO: 30) has an anionic warhead.

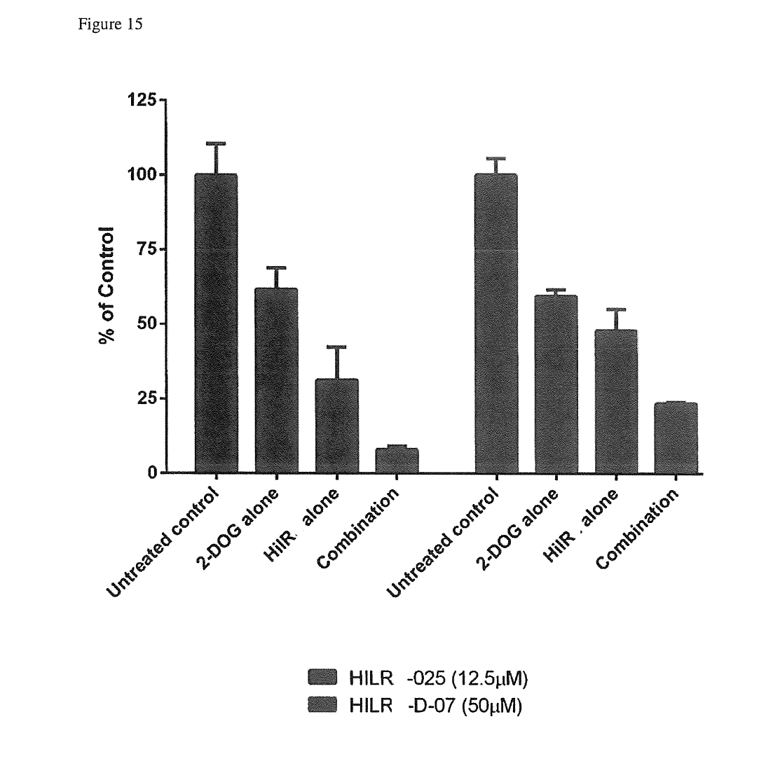

NCI-H460 human non-small-cell lung cancer cells were contacted with HILR-025 or HILR-D-07 alone or in combination with 3.125 mmol 2-DOG and cell survival was determined using AlamaBlue® cell viability reagent (Life Technologies, Inc.) in accordance with the manufacturer's instructions. The results of these studies are shown in FIG. 15.

The cell killing ability of both HILR-025 and HILR-D07 was found to be enhanced by co-administration with 2-DOG. 2-DOG is well tolerated in vivo and could be used to enhance the activity of the cyclic peptides disclosed herein. The similar results obtained for HILR-025 and HILR-D-07 suggests that these peptides have related mechanisms of action.

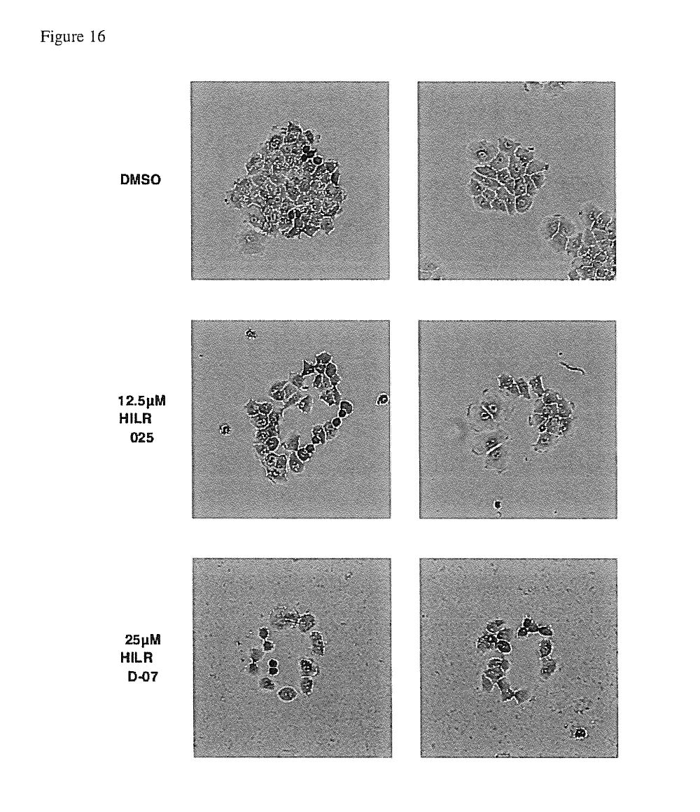

To investigate further the mechanism of action of the anionic warhead, cultures of NCI H460 Human Non-small cell lung cancer were exposed to HILR-025 and HILR-D-07 and observed using light microscopy. A comparative cell culture was treated with DMSO to provide a negative control. Light micrographs of the cell cultures are shown in FIG. 16.

Marked morphological changes were observed in the cell cultures exposed to cyclic compounds in accordance with the present disclosure. Ring-shaped morphology was observed which was comparable to that reported to the caused by THR53 in Warenius et al, Molecular Cancer (2011), 10:72-88. This suggests that THR53, HILR-025 and HILR-D-07 may have related mechanisms of action.

### Example 7. Effect of THR Cyclic Peptides HILR-025 and HILR-030 on the Activity of Lactate Dehydrogenase A [LDHA]

LDHA converts pyruvate to lactate with the production of one molecule of NAD (see FIG. 18). This NAD re-enters the Embden/Meyrhof pathway at the glyceraldehyde phosphate dehydrogenase step at which there is production of ATP. Without NAD this step in the anaerobic glycolysis pathway cannot occur and the cancer cell which relies predominantly on this pathway is deprived of the energy rich ATP molecule. For this reason two cyclic peptides, HILR-025 and HILR-030 were investigated as possible inhibitors of LDH activity.

An LDH activity assay was conducted on samples derived from NCI-H460 cells treated with 2 test compounds (HILR-025 and HILR-030) for either 24 h or 96 h. Significant cell death was observed at higher concentrations of test compounds, particularly at the later time point. Therefore a BCA assay was conducted to estimate the total amount of protein present in each LDH assay lysate and this was used to normalise the enzyme activity data. As an indication of cell viability, an Alamar blue assay was also carried out at both timepoints, to serve as an additional point of reference.

The following protocol was used:

1) NCI-H460 cells were grown in Ham's F12 media supplemented with 10% FBS.
2) Cells were harvested and seeded into 96-well plates at either 500 cells/well (for the 96 h timepoint) or 5000 cells/well for the 24 h timepoint.
3) Hilros compounds were made up from DMSO stock solutions and added directly to cells at concentrations of 40, 20, 10, 5 and 2.5 μM.
4) Parallel plates were set up:
For the LDH assay 10 replicates wells per assay concentration were used.
Triplicate wells were used for Alamar Blue assays
The final DMSO concentration in all wells was 0.2%.
   5) Cells were grown with compound for 24 or 96 hours at 37° C. 5% CO₂ in a humidified atmosphere.
   6) At the end of the assay (24 or 96 h), Alamar blue 10% (v/v) was added to one set of plates, incubated for a further 4 hours, and fluorescent product detected using the BMG FLUOstar plate reader.
   7) For the LDH assay, cells were harvested from each well by trypsinisation, cells from replicate wells pooled and then pelleted by centrifugation.
   8) Cell pellets were rinsed with ice-cold PBS, resuspended in 150 μl LDH assay buffer (provided in the kit) and snap frozen in liquid nitrogen to promote cell lysis.
   9) Samples were rapidly defrosted, and cell lysates cleared by centrifugation at 10,000×g for 10 min at 4° C.
   10) LDH activity was measured in the cleared lysates using an LDH activity kit (Abeam, ab102526).
   11) After preparation of the LDH activity assay reactions, according to the manufacturer's instructions, absorbance at 450 nm was measured at the initial time to determine (A450)initial
   12) Further absorbance readings were taken at 3 minute intervals for up to 15 minutes.

13) The final measurement [(A450)final] for calculating the enzyme activity was taken from the penultimate time point reading from when the most active sample exceeded the linear range of the standard curve.

14) The change in measurement from Tinitial to Tfinal for each sample was calculated: 11A450=(A450)final−(A450)initial
15) The NADH standard curve was used to interpolate the 11A450 for each sample to determine the amount of NADH generated by the kinase assay between Tinitial and Tfinal (B).
16) The LDH activity of each sample was determined by the following equation:

$$LDH Activity = \frac{B \times Sample Dilution Factor}{( Reaction Time ) \times V}$$

$$B = Amount ( nmole ) of NADH generated between {T}_{initial} and {T}_{final} . \\ Reaction Time = {T}_{final} - {T}_{initial} ( minutes )$$

$$V = sample volume ( mL ) added to well$$

a. Protein content in remaining cleared lysates was determined using a BCA assay (ThermoScientific).
b. Data were analysed using GraphPad Prism.

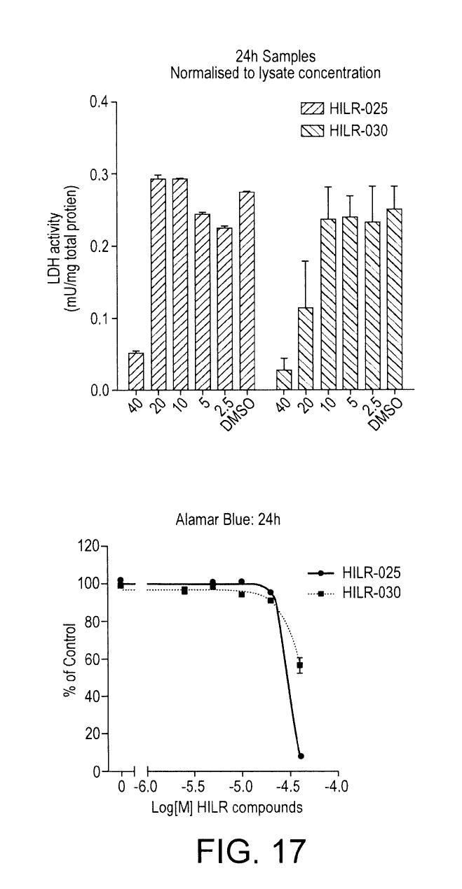

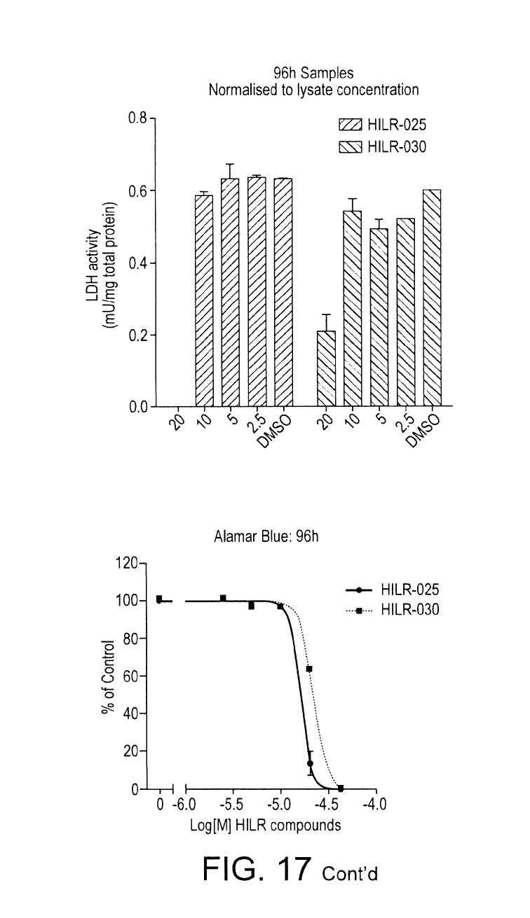

Results of the above assays are shown in FIG. 17. The data show that HILR-025 and HILR-030 are effective in inhibiting the activity of LDH, with HILR-025 having an IC₅₀ of 16 μM and HILR-030 having an IC₅₀ of 22 μM. This suggests that the cyclic peptides of the present invention target additionally the anaerobic glycolysis pathway of cancer cells.

LDH activity is typically expressed in milliunit/ml. One unit of LDH activity is defined as the amount of enzyme that catalyses the conversion of lactate into pyruvate to generate 1.0 μmole of NADH per minute at 37° C., therefore 1 mU/ml=1 nmole/min/ml. LDH activity data from this study is presented in the mU/ml format and also normalised to the total protein concentration of each lysate (mU/mg). Cell viability was monitored in parallel using Alamar Blue.

1-60. (canceled)

61. A compound capable of modulating the activity of poly(ADP-ribose) polymerase 1 (PARP-1) and/or lactate dehydrogenase A (LDHA), wherein the compound comprises a moiety according to a Formula 1 or salt, derivative, prodrug or mimetic thereof:
[X1-X2-X3-X4-X3-X4-X3-] Formula 1:
wherein X1 is a peptidic moiety capable of inhibiting the cleavage of PARP-1;
wherein X2 may be absent or present; when X2 is present, X2 is selected from Val or Ser;
wherein one of X3 and X4 is selected from Trp-Trp and Ar1-Ar2;
wherein the other of X3 and X4 is selected from Arg-Arg, Gpa-Gpa, Hca-Hca, and Ar3-Ar4; and
wherein
Hca represents the amino acid residue of homocysteic acid;
Gpa represents the amino acid residue of guanidinophenylalanine;
Ar1, Ar2, Ar3 and Ar4 each represent an amino acid residue having an aryl side chain, wherein the aryl side chains are independently selected from an optionally-substituted napthyl group, an optionally substituted 1,2-dihydronapthyl group, and an optionally-substituted 1,2,3,4-tetrahydronapthyl group; and
Aza represents the amino acid residue of azido-homoalanine.

62. The compound of claim 61, comprising at least one labelling moiety.

63. The compound of claim 62, wherein X1 is selected from SEQ ID NO: 21 (Formula 2), SEQ ID NO: 22 (Formula 3), SEQ ID NO: 23 (Formula 4) and SEQ ID NO: 24 (Formula 5):
-Pro-X5-X6-Pro-X7-Pro- SEQ ID NO: 21 (Formula 2):
wherein both X5 and X7 are amino acid residues bearing acidic side chains or wherein both X5 and X7 are amino acid residues bearing basic side chains;
wherein the amino acid residues bearing acidic side chains are each independently selected from Glu, Aza and Hca; and
wherein X6 is selected from Gly, Ala, MeGly and (CH₂)₃;
-Pro-X8-Gly-Pro-X9-Pro- SEQ ID NO: 22 (Formula 3):
wherein X8 and X9 are each independently selected from Asp and Glu;
-Pro-Arg-Lys-Pro-Arg-Pro-; SEQ ID NO: 23 (Formula 4):
-Gly-X11-Glu-Val-X12-X13- SEQ ID NO: 24 (Formula 5):
wherein X11 is selected from Asp and Glu;
wherein X12 is selected from Asp, an N-alkyl aspartic acid residue, and N-aryl aspartic acid residue Glu, an N-alkyl glutamic acid residue and an N-aryl glutamic acid residue;
wherein X13 is selected from Gly, an N-alkyl glycine residue, and an N-aryl glycine residue;
with the proviso that if X12 is Asp, X13 is an N-alkyl glutamic acid residue or an N-aryl glutamic acid residue.

64. The compound of claim 63, wherein X1 is of SEQ ID NO: 21 (Formula 2).

65. The compound of claim 64, wherein X5 is Glu or Hca and/or X7 is Glu or Hca.

66. The compound of claim 64, wherein X1 is selected from:
i.
SEQ ID NO: 2
-Pro-Arg-Gly-Pro-Arg-Pro-;
ii.
SEQ ID NO: 4
-Pro-Glu-Gly-Pro-Glu-Pro-;
iii.
SEQ TD NO: 25
-Pro-Hca-Gly-Pro-Hea-Pro-;
iv.
SEQ ID NO: 26
-Pro-Hca-MeGly-Pro-Hca-Pro-;
v.
SEQ ID NO: 27
-Pro-Aza-MeGly-Pro-Aza-Pro-;
vi.
SEQ ID NO: 28
-Pro-Hca-Gly-Pro-Aza-Pro-;
vii.
SEQ ID NO: 41
-Pro-Aza-Gly-Pro-Hca-Pro-;
and
viii.
SEQ ID NO: 42
-Pro-Aza-Gly-Pro-Aza-Pro.

67. The compound of claim 66, wherein X1 is of SEQ ID NO: 22 (Formula 3), X8 is Asp and X9 is Asp; or wherein X1 is of SEQ ID NO: 24 (Formula 5).

68. The compound of claim 63, wherein X1 is of SEQ ID NO: 24 (Formula 5), X11 is Asp and X12 is Asp or an N-alkyl aspartic acid residue.

69-76. (canceled)

77. A compound for use in modulating the activity of poly(ADP-ribose) polymerase 1 (PARP-1) and/or lactate dehydrogenase A (LDHA), which compound comprises a moiety according to Formula 6:
-Pro-X14-X15-Pro-X16-Pro- Formula 6:
wherein X14 and X16 are each amino acid residues bearing a side-chain, wherein each side-chain comprises an acidic functional group; and
wherein X15 is selected from Gly, Ala, MeGly, and (CH₂)₃.

78.-89. (canceled)

90. The compound of or a pharmaceutical composition comprising the compound of claim 61, wherein the compound or composition is for use in medicine for the treatment of cancer.

91. The compound of or a pharmaceutical composition comprising the compound of claim 61 for use in medicine for the treatment of cancer, wherein the compound or composition is to be administered with an aerobic glycolysis inhibitor.

92. The compound of or a pharmaceutical composition comprising the compound of claim 77, wherein the compound or composition is for use in medicine for the treatment of cancer.

93. The compound of or a pharmaceutical composition comprising the compound of claim 77 for use in medicine for the treatment of cancer, wherein the compound or composition is to be administered with an aerobic glycolysis inhibitor.

94.-117. (canceled)

118. A compound for the treatment of cancer comprising a poly(ADP-ribose) polymerase 1 (PARP-1) agonist and lactate dehydrogenase A (LDHA) inhibitor.

119. The compound according to claim 118, wherein the PARP-1 agonist and LDHA inhibitor is a single therapeutic agent.

120. The compound according to claim 118, wherein the compound is capable of binding to and/or protecting the DEVD or GDEVDG region of PARP-1 from cleavage.

121. The compound according to claim 118, wherein the compound comprises a peptide having between 16 and 18 amino acids or a salt, derivative, prodrug or mimetic thereof.

122. The compound according to claim 121, comprising the amino acid sequence of SEQ ID NO: 15 or SEQ ID NO: 16.

123. The compound according to claim 121, wherein the peptide comprises a 4 to 6 amino acid sequence which binds to the DEVD or GDEVDG region of PARP-1 and/or inhibits PARP cleavage.

124. (canceled)

125. The compound as claimed in claim 118, comprising or further comprising an aerobic glycolysis inhibitor that comprises 2-deoxyglucose (2-DOG).

126-160. (canceled)


SEQUENCE LISTING (42 sequences)

SEQ ID NO: 1 - protein, 6 aa, Homo sapiens
GDEVDG

SEQ ID NO: 2 - protein, 6 aa, Artificial Sequence
  cancerocidal group
PRGPRP

SEQ ID NO: 3 - protein, 6 aa, Artificial Sequence
  comparative peptide
PRRPGP

SEQ ID NO: 4 - protein, 6 aa, Artificial Sequence
  comparative peptide
PEGPEP

SEQ ID NO: 5 - protein, 6 aa, Artificial Sequence
  Partial sequence of a Jun binding peptide
PRKPRP

SEQ ID NO: 6 - protein, 18 aa, Artificial Sequence
  Cyclic peptide
  MISC_FEATURE (1)..(18): cyclic peptide
AAAGGGPRGPRPGGGAAA

SEQ ID NO: 7 - protein, 18 aa, Artificial Sequence
  cyclic peptide
  MISC_FEATURE (1)..(18): cyclic peptide
GGGGGGPRGPRPGGGGGG

SEQ ID NO: 8 - protein, 18 aa, Artificial Sequence
  cyclic peptide
  MISC_FEATURE (1)..(18): cyclic peptide
GGGGGGPRGPRPGGGGGG

SEQ ID NO: 9 - protein, 18 aa, Artificial Sequence
  cyclic peptide
  MISC_FEATURE (1)..(18): cyclic peptide
AAGPGGPRGPRPGGPGAA

SEQ ID NO: 10 - protein, 10 aa, Artificial Sequence
  cassette
ALKLALKLAL

SEQ ID NO: 11 - protein, 19 aa, Artificial Sequence
  cyclic peptide
  MISC_FEATURE (1)..(19): cyclic peptide
PRGPRPVKLALKLALKLAL

SEQ ID NO: 12 - protein, 18 aa, Artificial Sequence
  cyclic peptide
  MISC_FEATURE (1)..(18): cyclic peptide
PRGPRPVKLALKLALKFP

SEQ ID NO: 13 - protein, 17 aa, Artificial Sequence
  cyclic peptide
  MISC_FEATURE (1)..(17): cyclic peptide
PRGPRPVALKLALKLAL

SEQ ID NO: 14 - protein, 18 aa, Artificial Sequence
  cyclic peptide
  MISC_FEATURE (1)..(18): cyclic peptide
PRGPRPVALKLALKALAL

SEQ ID NO: 15 - protein, 17 aa, Artificial Sequence
  cyclic peptide
  MISC_FEATURE (1)..(17): cyclic peptide
PRGPRPVWWRRWWRRWW

SEQ ID NO: 16 - protein, 17 aa, Artificial Sequence
  cyclic peptide
  MISC_FEATURE (1)..(17): cyclic peptide
  MOD_RES (10)..(10): Guanidinophenylalanine
  MOD_RES (11)..(11): Guanidinophenylalanine
  MOD_RES (15)..(15): guanidinophenylalanine
  MOD_RES (16)..(16): guanidinophenylalanine
PRGPRPVWWXXWWXXXW

SEQ ID NO: 17 - protein, 10 aa, Artificial Sequence
  cassette
WWRRWWRRWW

SEQ ID NO: 18 - protein, 10 aa, Artificial Sequence
  cassette
  MOD_RES (3)..(3): guanidinophenylalanine
  MOD_RES (4)..(4): guanidinophenylalanine
  MOD_RES (7)..(7): guanidinophenylalanine
  MOD_RES (8)..(8): guanidinophenylalanine
WWXXWWXXWW

SEQ ID NO: 19 - protein, 17 aa, Artificial Sequence
  cyclic peptide
  MISC_FEATURE (1)..(17): cyclic peptide
PEGPEPVWWRRWWRRWW

SEQ ID NO: 20 - protein, 17 aa, Artificial Sequence
  cyclic peptide
  MISC_FEATURE (1)..(17): cyclic peptide
  MOD_RES (2)..(2): homocysteic acid
  MOD_RES (5)..(5): homocysteic acid
PXGPXPVWWRRWWRRWW

SEQ ID NO: 21 - protein, 6 aa, Artificial Sequence
  cancerocidal group
  MISC_FEATURE (2)..(2): basic residue, or an acidic residue selected from homocysteic acid azidohomoalanine and glutamic acid
  MISC_FEATURE (3)..(3): selected from Gly, Ala, MeGly, and (CH2)3
  MISC_FEATURE (5)..(5): If residue 2 is acidic, an acidic residue selected from glutamic acid and homocysteic acid. If residue 2 is basic, a basic residue.
PXXPXP

SEQ ID NO: 22 - protein, 6 aa, Artificial Sequence
  cancerocidal group
  MISC_FEATURE (2)..(2): selected from Asp and Glu.
  MISC_FEATURE (5)..(5): selected from Asp and Glu.
PXGPXP

SEQ ID NO: 23 - protein, 6 aa, Artificial Sequence
  cancerocidal group
PRKPRP

SEQ ID NO: 24 - protein, 6 aa, Artificial Sequence
  cancerocidal group
  MISC_FEATURE (2)..(2): selected from Asp and Glu
  MISC_FEATURE (5)..(5): selected from Asp, N-alkyl Asp, N-aryl Asp, Glu, N-alkyl Glu, N-Aryl Glu.
  MISC_FEATURE (6)..(6): selected from Gly, N-alkyl Gly, N-aryl Gly. If residue 5 is Asp, residue 6 is not Gly.
GXEVXX

SEQ ID NO: 25 - protein, 6 aa, Artificial Sequence
  cancerocidal group
  MOD_RES (2)..(2): Homocysteic acid
  MOD_RES (5)..(5): Homocysteic acid
PXGPXP

SEQ ID NO: 26 - protein, 6 aa, Artificial Sequence
  cancerocidal group
  MOD_RES (2)..(2): homocysteic acid
  MOD_RES (3)..(3): MeGly
  MOD_RES (5)..(5): homocysteic acid
PXGPXP

SEQ ID NO: 27 - protein, 6 aa, Artificial Sequence
  cancerocidal group
  MOD_RES (2)..(2): Azidohomoalanine
  MOD_RES (3)..(3): MeGly
  MOD_RES (5)..(5): Azidohomoalanine
PXGPXP

SEQ ID NO: 28 - protein, 6 aa, Artificial Sequence
  cancerocidal group
  MOD_RES (2)..(2): Homocysteic acid
  MOD_RES (5)..(5): Azidohomoalanine
PXGPXP

SEQ ID NO: 29 - protein, 7 aa, Artificial Sequence
  cancerocidal group
  MOD_RES (5)..(5): N-methyl aspartic acid
  MOD_RES (6)..(6): MeGly
GDEVDGV

SEQ ID NO: 30 - protein, 17 aa, Artificial Sequence
  cyclic peptide
  MISC_FEATURE (1)..(17): cyclic peptide
  MOD_RES (2)..(2): Homocysteic acid
  MOD_RES (5)..(5): homocysteic acid
  MOD_RES (10)..(10): guanidinophenylalanine
  MOD_RES (11)..(11): guanidinophenylalanine
  MOD_RES (14)..(14): guanidinophenylalanine
  MOD_RES (15)..(15): guanidinophenylalanine
PXGPXPVWWXXWWXXWW

SEQ ID NO: 31 - protein, 17 aa, Artificial Sequence
  cyclic peptide
  MISC_FEATURE (1)..(17): cyclic peptide
  MOD_RES (2)..(2): homocysteic acid
  MOD_RES (5)..(5): homocysteic acid
  MOD_RES (8)..(8): residue of 3-amino-3-(-2-napthyl)-propionic acid
  MOD_RES (9)..(9): residue of 3-amino-3-(-2-napthyl)-propionic acid
  MOD_RES (12)..(12): residue of 3-amino-3-(-2-napthyl)-propionic acid
  MOD_RES (13)..(13): residue of 3-amino-3-(-2-napthyl)-propionic acid
  MOD_RES (16)..(16): residue of 3-amino-3-(-2-napthyl)-propionic acid
  MOD_RES (17)..(17): residue of 3-amino-3-(-2-napthyl)-propionic acid
PXGPXPSXXRRXXRRXX

SEQ ID NO: 32 - protein, 17 aa, Artificial Sequence
  cyclic peptide
  MISC_FEATURE (1)..(17): cyclic peptide
  MOD_RES (8)..(8): Residue of glutamic acid-gamma-[2-(1-sulfonyl-5-napthyl)-aminoethylamide
  MOD_RES (9)..(9): Residue of glutamic acid-gamma-[2-(1-sulfonyl-5-napthyl)-aminoethylamide
  MOD_RES (12)..(12): Residue of glutamic acid-gamma-[2-(1-sulfonyl-5-napthyl)-aminoethylamide
  MOD_RES (13)..(13): Residue of glutamic acid-gamma-[2-(1-sulfonyl-5-napthyl)-aminoethylamide
  MOD_RES (16)..(16): Residue of glutamic acid-gamma-[2-(1-sulfonyl-5-napthyl)-aminoethylamide
  MOD_RES (17)..(17): Residue of glutamic acid-gamma-[2-(1-sulfonyl-5-napthyl)-aminoethylamide
PRGPRPVXXRRXXRRXX

SEQ ID NO: 33 - protein, 17 aa, Artificial Sequence
  cyclic peptide
  MISC_FEATURE (1)..(17): cyclic peptide
  MOD_RES (2)..(2): homocysteic acid
  MOD_RES (5)..(5): Azidohomoalanine
PXGPXPVWWRRWWRRWW

SEQ ID NO: 34 - protein, 17 aa, Artificial Sequence
  cyclic peptide
  MISC_FEATURE (1)..(17): cyclic peptide
  MOD_RES (2)..(2): homocysteic acid
  MOD_RES (5)..(5): homocysteic acid
  MOD_RES (8)..(8): residue of 3-amino-3-(-2-napthyl)-propionic acid
  MOD_RES (9)..(9): residue of 3-amino-3-(-2-napthyl)-propionic acid
  MOD_RES (10)..(10): homocysteic acid
  MOD_RES (11)..(11): homocysteic acid
  MOD_RES (12)..(12): residue of 3-amino-3-(-2-napthyl)-propionic acid
  MOD_RES (13)..(13): residue of 3-amino-3-(-2-napthyl)-propionic acid
  MOD_RES (14)..(14): homocysteic acid
  MOD_RES (15)..(15): homocysteic acid
  MOD_RES (16)..(16): residue of 3-amino-3-(-2-napthyl)-propionic acid
  MOD_RES (17)..(17): residue of 3-amino-3-(-2-napthyl)-propionic acid
PXGPXPVXXXXXXXXXX

SEQ ID NO: 35 - protein, 17 aa, Artificial Sequence
  cyclic peptide
  MISC_FEATURE (1)..(17): cyclic peptide
  MOD_RES (2)..(2): homocysteic acid
  MOD_RES (5)..(5): azidohomoalanine
  MOD_RES (8)..(8): residue of 3-amino-3-(-2-napthyl)-propionic acid
  MOD_RES (9)..(9): residue of 3-amino-3-(-2-napthyl)-propionic acid
  MOD_RES (10)..(10): homocysteic acid
  MOD_RES (11)..(11): homocysteic acid
  MOD_RES (12)..(12): residue of 3-amino-3-(-2-napthyl)-propionic acid
  MOD_RES (13)..(13): residue of 3-amino-3-(-2-napthyl)-propionic acid
  MOD_RES (14)..(14): homocysteic acid
  MOD_RES (15)..(15): homocysteic acid
  MOD_RES (16)..(16): residue of 3-amino-3-(-2-napthyl)-propionic acid
  MOD_RES (17)..(17): residue of 3-amino-3-(-2-napthyl)-propionic acid
PXGPXPVXXXXXXXXXX

SEQ ID NO: 36 - protein, 17 aa, Artificial Sequence
  cyclic peptide
  MISC_FEATURE (1)..(17): cyclic peptide
  MOD_RES (2)..(2): azidohomoalanine
  MOD_RES (3)..(3): MeGly
  MOD_RES (5)..(5): azidohomoalanine
  MOD_RES (8)..(8): residue of 3-amino-3-(-2-napthyl)-propionic acid
  MOD_RES (9)..(9): residue of 3-amino-3-(-2-napthyl)-propionic acid
  MOD_RES (10)..(10): homocysteic acid
  MOD_RES (11)..(11): homocysteic acid
  MOD_RES (12)..(12): residue of 3-amino-3-(-2-napthyl)-propionic acid
  MOD_RES (13)..(13): residue of 3-amino-3-(-2-napthyl)-propionic acid
  MOD_RES (14)..(14): homocysteic acid
  MOD_RES (15)..(15): homocysteic acid
  MOD_RES (16)..(16): residue of 3-amino-3-(-2-napthyl)-propionic acid
  MOD_RES (17)..(17): residue of 3-amino-3-(-2-napthyl)-propionic acid
  MOD_RES (17)..(17): residue of 3-amino-3-(-2-napthyl)-propionic acid
PXGPXPVXXXXXXXXXX

SEQ ID NO: 37 - protein, 6 aa, Artificial Sequence
  cancerocidal group
  MISC_FEATURE (2)..(2): Any natural or unnatural amino acid bearing an acidic side chain.
  MISC_FEATURE (3)..(3): Selected from Gly, Ala, MeGly and (CH2)3
  MISC_FEATURE (5)..(5): Any natural or unnatural amino acid bearing an acidic side-chain.
PXXPXP

SEQ ID NO: 38 - protein, 16 aa, Artificial Sequence
  cassette
RRWRRWWRRWWRRWRR

SEQ ID NO: 39 - protein, 8 aa, Artificial Sequence
  cassette
  MISC_FEATURE (1)..(8): cyclic peptide
WRWRWRWR

SEQ ID NO: 40 - protein, 17 aa, Artificial Sequence
  cyclic peptide
  MISC_FEATURE (1)..(17): cyclic peptide
  MOD_RES (5)..(5): N-methyl aspartic acid
  MOD_RES (6)..(6): MeGly
GDGVDGVWWRRWWRRWW

SEQ ID NO: 41 - protein, 6 aa, Artificial Sequence
  cancerocidal group
  MOD_RES (2)..(2): Azidohomoalanine
  MOD_RES (5)..(5): Homocysteic acid
PAGPEP

SEQ ID NO: 42 - protein, 6 aa, Artificial Sequence
  cancerocidal group
  MOD_RES (2)..(2): Azidohomoalanine
  MOD_RES (5)..(5): Azidohomoalanine
PAGPAP